SEQUENCE LISTING (3 sequences)

SEQ ID NO: 1 - protein, 499 aa, Homo sapiens
MAQSTATSPDGGTTFEHLWSSLEPDSTYFDLPQSSRGNNEVVGGTDSSMDVFHLEGMTTSVMAQFNLLSSTMDQMSSRAASASPYTPEHAASVPTHSPYAQPSSTFDTMSPAPVIPSNTDYPGPHHFEVTFQQSSTAKSATWTYSPLLKKLYCQIAKTCPIQIKVSTPPPPGTAIRAMPVYKKAEHVTDVVKRCPNHELGRDFNEGQSAPASHLIRVEGNNLSQYVDDPVTGRQSVVVPYEPPQVGTEFTTILYNFMCNSSCVGGMNRRPILIIITLEMRDGQVLGRRSFEGRICACPGRDRKADEDHYREQQALNESSAKNGAASKRAFKQSPPAVPALGAGVKKRRHGDEDTYYLQVRGRENFEILMKLKESLELMELVPQPLVDSYRQQQQLLQRPSHLQPPSYGPVLSPMNKVHGGMNKLPSVNQLVGQPPPHSSAATPNLGPVGPGMLNNHGHAVPANGEMSSSHSAQSMVSGSHCTPPPPYHADPSLVRTWGP

SEQ ID NO: 2 - protein, 393 aa, Homo sapiens [sequence omitted]

SEQ ID NO: 3 - protein, 391 aa, Artificial
  p53 chimera comprising p73 oligomerization domain
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQKRRHGDEDTYYLQVRGRENFEILMKLKESLELMELVPQPLVDSYRHSSHLKSKKGQSTSRHKKLMFKTEGPDSD

(19) United States
(12) Patent Application Publication
(10) Pub. No.: US 2013/0011333 A1
(43) Pub. Date: Jan. 10, 2013

(54) Methods and Compositions for Nanoparticle-Mediated Cancer Cell-Targeted Delivery

(75) Inventors: Zhi-Min Yuan, Newton, MA (US); Seog-Jin Seo, Cheongwon-gun (KR)

(73) Assignee: Board of Regents, The University of Texas System, Austin, TX (US)

(21) Appl. No.: 13/522,907

(22) PCT Filed: Jan. 18, 2011
(86) PCT No.: PCT/US11/21547

§ 371 Date: Sep. 25, 2012

Related U.S. Application Data
(60) Provisional application No. 61/295,898, filed on Jan. 18, 2010.

(51) Int. Cl. A61K 9/14; A61K 38/02; A61K 51/12; C12N 5/071; C12Q 1/02; A61K 51/04; A61K 49/00; A61N 5/10; A61K 31/7088; A61P 35/00; B82Y 5/00

(52) U.S. Cl. 424/1.29; 514/44 R; 424/493; 514/44 A; 514/1.1; 435/375; 514/19.3; 435/29; 424/1.11; 424/9.1; 600/1; 977/773; 977/915

(57) ABSTRACT

The present invention provides methods and compositions to selectively and directly deliver nanoparticles carrying an active agent to tumor cells. The active agent is internalized by the tumor cells, producing an anti-tumor effect for therapeutic applications and/or depositing a detectable marker for diagnostic applications. The present invention further provides a p53 chimera that circumvents the dominant negative activity of mutant p53 as a therapeutic in the treatment of cancer and reduction of mor size.

### STATEMENT OF PRIORITY

This application claims the benefit, under 35 U.S.C. §119(e), of U.S. Provisional Application Ser. No. 61/295,898, filed Jan. 18, 2010, the entire contents of which are incorporated herein by reference.

### FIELD OF THE INVENTION

The present invention relates to compositions and methods for selectively delivering an active agent to a cancer cell via a nanoparticle.

### BACKGROUND OF THE INVENTION

The effectiveness of anticancer therapeutics relies on the ability to reduce and eliminate cancer cells without damaging healthy tissues. Thus, a strategy of preferentially targeting cancer cells is essential in the success of cancer therapeutics. Nanoparticle-based systems have been explored to enhance delivery of therapeutic agents to cancer cells. While new developments have improved drug delivery efficiency, it remains a huge challenge to attain cancer-cell specific delivery.

A cancer cell phenomenon known as the Warburg effect defines the well characterized tendency of cancer cells to eagerly import glucose into the cell to fuel the run-away metabolic activity characteristic of malignancies. The most familiar use of the Warburg effect is in positron-emission tomography (PET) whereby a radionuclide is attached to a glucose molecule and administered to a patient. Malignant cells take up much more of the glucose-radionuclide medium, which is internalized into the cell, or at least attached to the tumor cell. The radiation being emitted by the radionuclide results in the release of photons at the site of the tumor, which in turn are detected by the PET (photon) detection machine. Thus, PET scans are a very accurate and specific method for detecting cancers.

The present invention overcomes previous shortcomings in the art by exploiting the Warburg effect to target active agents directly and selectively to cancer cells via nanoparticles, to deliver therapeutics (e.g., drugs, nucleic acids, proteins/peptides) and/or position a detectable marker for diagnosis.

In further aspects, the tumor suppressor p53 is a transcription factor that primarily binds as a tetramer to the p53 responsive element within the promoter of a host of genes, whose products are involved in control of growth arrest, apoptosis, senescence, differentiation and DNA repair-cellular events that are critical for maintenance of genomic stability and homeostasis [1, 2]. p53 is activated by a variety of stress signals, including oncogene activation and DNA damage and post-translational modifications have been shown to be the major mechanisms of p53 activation, which result in an increase of p53 protein abundance and transcription activity [1, 2].

Because of its growth inhibitory activities, the level of wild type p53 is usually kept low under normal physiological conditions, which is chiefly mediated via regulation of p53 protein stability. While a number of regulators have been reported to be involved, MDM2 is the principal player in control of p53 turnover. MDM2 is an E3 ubiquitin ligase that targets p53 for ubiquitination and subsequent degradation but at the same time is a transcriptional target of p53, which thereby creates a negative regulatory loop. Various stress signals all impinge on this regulatory loop to impact p53 activity [1, 2]. In contrast to wild-type p53, mutant p53 proteins usually accumulate in cancer cells at a high level, which has been attributed to the inability of mutant p53 to induce MDM2 expression. Indeed, a genetic study using a mutant p53 knock-in mouse demonstrated that mutant p53 protein levels are mainly regulated by MDM2 [3].

The crucial role of p53 as a tumor suppressor is supported by its universal inactivation in cancer cells either through mutations affecting the p53 locus directly or through aberration of its normal regulation [1, 2]. p53 mutations have been identified in almost all types of human cancers at various frequencies, ranging from ˜10% (for example, in hematopoietic malignancies [4]) to 50-70% (for example, in ovarian [5], colorectal [6] and head and neck [7] cancers). Ample evidence indicates that while wild-type p53 confers cancer cells sensitivity to chemo and radiotherapies, mutant p53 in cancer cells is responsible for therapy resistance and poor prognosis. Correlation between expression of mutant p53 and cancer therapy resistance has been widely reported. For instance, resistance to cisplatin has been observed in ovarian cancer patients [8]. Colorectal cancers were also demonstrated to be less chemosensitive in the presence of mutant p53 [9]. Similarly, p53 inactivation conferred hematological malignancies resistance to anti-neoplastic drugs [10].

p53 mutations are unique in that the majority of p53 alterations are missense mutations that lead to the synthesis of a full-length protein [11]. While most p53 mutations are found within the core DNA-binding domain (DBD) resulting in loss of sequence-specific DNA-binding activity, there is a high degree of structural, biochemical, and biological heterogeneity. Most p53 mutations can be classified into two main categories according to their effect on the thermodynamic stability of the p53 protein [12], commonly referred to as “DNA-contact” and “conformational” mutations. The DNA-contact group includes mutations in residues directly involved in DNA binding, such as R248Q and R273H. This group has a wild-type conformation as probed by conformational monoclonal antibodies and does not bind to the chaperone hsp70 [13, 14]. The conformational group comprises mutations that cause local (such as R249S and G245S) or global (such as R175H and R282W) conformational distortions that result in proteins with intense binding to hsp70. The conformational mutations are associated with a more severe phenotype in vitro than the DNA contact mutations [13]. The heterogeneity of p53 mutations can also be reflected in the nature of the resulting residue. Mutant R273H has a wild type conformation whereas mutant R273P is denatured [13].

Apart from losing the tumor suppressor function, mutant p53 acquires a dominant-negative activity over wild-type p53 through hetero-oligomerization between mutant and wild type p53, which is a very effective mechanism of p53 inactivation since only one molecule of mutant p53 within a tetramer can significantly compromise the DNA binding [1, 2]. In addition, studies have shown that mutant p53 can gain new oncogenic activities independent of wild type p53 [15-17]. While unified mechanisms underlying the gain-of-function properties of p53 remain to be elucidated, a number of models have been proposed. Mutant p53 has been shown to interact with p63 and p73 via their DNA binding domains. This interaction leads to inactivation of p73 and p63 function [18-21]. A genetic study with knock-in mice expressing the common p53 mutants (R248W and R273H) showed that the mutants acquired oncogenic properties by inactivation of ATM, a protein kinase critical for DNA damage response [22]. Collectively, available information indicates that mutant p53 contributes to tumorigenesis and therapy resistance via a combination of loss-of-function, dominant-negative and gain-of-function activities. Many efforts have been made to develop small molecules able to reactivate mutant p53. However, the structural heterogeneity of mutant p53 as reflected by more than 2000 different types of mutant p53 proteins in cancer cells [23] imposes a significant challenge for developing versatile p53-reactivators. An alternative approach is to reintroduce wild-type p53 back to tumor cells via gene therapy. Replication deficient virus-based vectors have demonstrated some therapeutic effects in head and neck cancers; however, the high level of mutant p53 proteins in tumor cells is the major hurdle in reinstating functional p53. The present invention overcomes these previous shortcomings in the art by providing a p53 derivative that does not bind to mutant p53 and thus can effectively restore p53 function in the presence of high level of p53 mutants.

### SUMMARY OF THE INVENTION

In one aspect, the present invention provides a nanoparticle selected from the group consisting of:

A) a nanoparticle comprising:
a) a polycation/polyalkylene glycol/glucose conjugate; and
b) an active agent;

B) a nanoparticle comprising:
a) a core comprising an active agent; and
b) a glucose/polyalkylene glycol conjugate surrounding the core of (a);

C) a nanoparticle comprising:
a) a core comprising a polycation and an active agent; and
b) a glucose/polyalkylene glycol conjugate surrounding the core of (a);

D) a nanoparticle comprising:
a) a polycation/alginate/glucose conjugate; and
b) an active agent;

E) a nanoparticle comprising:
a) a core comprising an active agent; and
b) a glucose/alginate conjugate surrounding the core of (a);

F) a nanoparticle comprising:
a) a core comprising a polycation and an active agent; and
b) a glucose/alginate conjugate surrounding the core of (a); and

G) any combination of the nanoparticles of (A)-(F) above.

In the nanoparticles of this invention, the active agent can be a polynucleotide, an oligonucleotide, an interfering RNA, a protein, a peptide, a chemotherapeutic drug, a cytotoxic agent, a radionuclide, a detectable marker, an imaging agent and any combination thereof.

In a particular embodiment of this invention, a nanoparticle is provided, comprising: a) a core comprising polyethylenimine (PEI) and a polynucleotide encoding p53; and b) a glucose/polyethyleneglycol (PEG) conjugate surrounding the core of (a).

In further embodiments, the present invention provides a nanoparticle, comprising a) a core comprising polyethylenimine (PEI) and a polynucleotide encoding a p53 chimera comprising a p73OD (e.g., p53/p73OD); and b) a glucose/polyethyleneglycol (PEG) conjugate surrounding the core of (a). Additionally provided herein is a composition comprising a p53 chimera comprising a p73OD in a pharmaceutically acceptable carrier.

Furthermore, the present invention provides a method of delivering a nanoparticle to a cell, comprising contacting the cell with a nanoparticle of this invention under conditions whereby the nanoparticle binds to a glucose transporter at the cell surface and is internalized by the cell, which cell can be in vivo or in vitro.

Additionally provided herein is a method of delivering an active agent to a tumor cell in a subject (e.g., a subject in need thereof), comprising delivering a nanoparticle of this invention to the subject, whereby the nanoparticle binds to a glucose transporter at the tumor cell surface and is internalized by the tumor cell, thereby delivering the active agent to the tumor cell.

In yet further embodiments, the present invention provides a method of decreasing the size of a tumor in a subject (e.g., a subject in need thereof), comprising delivering an effective amount of a nanoparticle of this invention to the subject, whereby the nanoparticle binds to a glucose transporter at the surface of a tumor cell and is internalized by the cell, thereby decreasing the size of the tumor in the subject.

The present invention further provides a method of treating cancer in a subject (e.g., a subject in need thereof), comprising delivering an effective amount of a nanoparticle of this invention to the subject, thereby treating cancer in the subject.

Furthermore, the present invention provides a method of delivering a p53 chimera comprising p73OD to a cell (e.g., a cancer cell or tumor cell), comprising contacting the cell with the p53 chimera of this invention under conditions whereby the p53 chimera is internalized by the cell, which cell can be in vivo or in vitro.

In yet further embodiments, the present invention provides a method of decreasing the size of a tumor in a subject (e.g., a subject in need thereof), comprising delivering an effective amount of a p53 chimera of this invention to the subject, whereby the chimera is internalized by cells of the tumor, thereby decreasing the size of the tumor in the subject.

The present invention further provides a method of treating cancer in a subject (e.g., a subject in need thereof), comprising delivering an effective amount of a p53 chimera of this invention to the subject, thereby treating cancer in the subject.

Further embodiments of the present invention include a method of inducing tumor regression in a subject in need thereof, comprising delivering an effective amount of a p53 chimera of this invention to the subject, thereby inducing tumor regression in the subject.

The methods described herein can further comprise the step of administering to the subject an inhibitor of protein kinase, an inhibitor of histone deacetylase (HDAC), an inhibitor of methyltransferase, and any combination thereof.

The methods described above can also comprise the step of administering to the subject a chemotherapeutic agent, an anti-angiogenic agent, a radiation treatment, a surgical treatment or any combination thereof. This further step can be carried out before, after and/or simultaneously with the delivery of the nanoparticle and/or p53 chimera to the subject.

Additional aspects of this invention include a composition comprising the nanoparticle of this invention in a pharmaceutically acceptable carrier, as well as a kit comprising the nanoparticle of this invention and/or the composition of this invention.

Also provided herein is an in vitro method of diagnosing cancer in a subject, comprising: a) contacting a nanoparticle of this invention with cells from the subject, wherein the nanoparticle comprises a detectable marker; b) measuring the rate and/or amount and/or specificity of internalization of the nanoparticles into the cells of the subject; and c) comparing the rate and/or amount and/or specificity of internalization of the nanoparticles into the cells of the subject with the rate and/or amount and/or specificity of internalization of the nanoparticles into cells of a control subject and/or of control cells of the subject being diagnosed, whereby an increase or change in the amount and/or rate and/or specificity of internalization of the nanoparticles into cells of the subject as compared with the cells of the control subject and/or the control cells is diagnostic of cancer in the subject.

Furthermore, the present invention provides an in vivo method of diagnosing cancer in a subject, comprising: a) delivering a nanoparticle of any of claims 1-6 to the subject, wherein the nanoparticle comprises an imaging agent; b) detecting a signal from the imaging agent in the subject; and c) comparing the signal from the imaging agent in the subject with the signal from the same imaging agent in a control subject and/or in control tissue/cells from the subject being diagnosed, whereby an alteration in the signal from the subject as compared with the signal from the control subject and/or control tissue/cells is diagnostic of cancer in the subject.

### BRIEF DESCRIPTION OF THE FIGURES

FIG. 1. Synthesis of a GLU-PEG-PEI plasmid complex.

FIGS. 2A-C. A. Differential expression of a plasmid encoding green fluorescent protein (GFP) in two cell lines, breast carcinoma cell line, MDA-MB-231 and a non-transformed human breast epithelial cell line, MCF-10A. B. Preferential targeting of nanoparticles in a prostate carcinoma cell line, PC3, as compared to a non-transformed prostate epithelial cell line, RWPE. C. Differential expression of a plasmid encoding beta galactosidase (β-gal) in two cancer cell lines and two untransformed cell lines.

FIGS. 3A-B. A. Competition assay demonstrating that GLU-PEG-PEI-mediated nucleic acid delivery is glucose-transporter dependent. B. Nanoparticle-nucleic acid complexes enter cells via endocytosis-mediated internalization.

FIGS. 4A-B. A. Tumor-bearing mice (tumor volume approximately 100 mm³) were intravenously injected with a tumor suppressor gene (PTEN, 20 or 40 μg) plasmid in complex with nanoparticles (50 μl total volume). The mice were sacrificed 14 days later and the tumors were collected. B. Tumor tissue sections were stained with Ki-67, a proliferation marker. Shown are representative images of Ki-67 staining and tumor size. The sequence for PTEN phosphatase and tensin homolog is provided under GenBank® Database Accession No. NM_{—}000314.4. (Li et al. “PTEN, a putative protein tyrosine phosphatase gene mutated in human brain, breast, and prostate cancer” Science 275(5308):1943-1947 (1997)).

FIGS. 5A-B. p53/73OD but not wild-type p53 suppresses growth of mutant p53 expressing cells. MDA-MB-231 cells were transfected with a pCNDA3 vector encoding control vector (1.0 μg), wild-type p53 (1.0 μg), p53/p73OD (0.5 μg) or p53(R175H)/p73OD (1.0 μg). After selection with puromycin, the cells were subjected to colony formation assay. The data are means±SE from three separate experiments (A). Western analysis was performed with cells that were harvested at 48 h pos-selection and probed using the indicated antibodies (B). Anti-Myc blot shows the levels of exogenous p53 and anti-p53 blot reflect the combined levels of endogenous mutant p53 and exogenous p53.

FIG. 6. MCF-10A cells are less sensitive to a moderate level of p53/p73OD expression. MCF-10A cells with MDA-MB-231 cells as controls were transfected with the indicated vectors (0.5 μg) and subjected to colony formation assay as in FIG. 5. Western blot was performed in parallel to determine the level of p53/p73OD expression.

FIGS. 7A-C. p53/p73OD expression resulted in different responses in MDA-MB-231 cells. A. Cells as in FIG. 6 were subject to Western analysis using the indicated antibodies. P53/p73OD expressing MDA-MB-231 or MCF-10A cells were either mock or irradiated with 4Gy. The cells were either harvested at 6 h post-IR for Western analysis (B) or at 24 h for FACS analysis (C). The results are mean±SE from three independent experiments.

FIGS. 8A-C. The Glu-PEG-PEI nanoparticles specifically target malignant cells for gene delivery. MCF-10A/MDA-MB-231(a) or RWPE/PC3 (B) cells were incubated with Glu-PEG-PEI/lacZ DNA complex. Cells were fixed 24 hours later and stained with X-gal® reagent. C. 50 mM glucose was included in culture medium for competing with the uptake of GLU-PEG-PEI-β-gal. Shown are representative images.

FIG. 9. GLU-ALG reduces the cytotoxicity of PEI. The effect of increasing concentration of PEI (0, 0.05, 0.1, 0.25, 0.5, 1.0, 2.5 or 5.0 μg/ml) on MDA-MB-231 cell viability was assessed by colony survival assay using a method as described in FIG. 5. The effect of GLU-ALG was examined by mixing 1 μg/ml of PEI with 0.05, 0.1, 0.25, 0.5, 1.0, 2.5 or 5.0 μg/ml of GLU-ALG.

FIG. 10. GLU-ALG-PEI mediated tumor-specific delivery of the LacZ expressing plasmid. Breast cancer cell line MDA-MB-231 (5 million cells) were implanted into the flank region of nude mice (4-6 weeks of age) and allowed two weeks of tumor development. Once the tumor size reached approximately 200 mm in volume, the mice were starved overnight. The next morning the LacZ expression plasmid was mixed with PEI followed by Glucose-Alginic acid and injected through i.v. Mice were sacrificed 48 h post injection. Different tissue samples including heart, lung, spleen, liver, kidney and tumor samples were harvested. Tissue sections were prepared and stained with β-Gal as described. Representative images from each tissue are shown.

FIG. 11. Induction of Pg13-GFP in MDA-MB-231 cells by expression of p53/p73OD. MDA-MB-231 cells stably expressing Pg13-GFp were incubated with either GLU-PEG-PEI/WTp53 or p53/p73OD [a low level of plasmid (0.2 μg DNA/60-mm dish) was used to avoid p53-mediated cell death]. The cells were fixed 24 h later using 2% formalin and stained with DAPI dye. The top panels show the GFP expression. The bottom panels show cell nuclei detected by DAPI staining.

FIG. 12. Schematic of p53, p7313 and p53 chimera. The p53 chimera comprises the oligomerization domain (OD) of p7313 where the 393 amino acid sequence of the p53 protein is substituted at amino acids 318 to 364 with amino acids 346-390 of the p73β protein. TAD: transactivation domain; PRD: proline rich domain; DBD: DNA binding domain; Oligo: oligomerization domain.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a biomolecule or agent of this invention, dose, time, temperature, and the like, is meant to encompass variations of ±20%, ±10%, ±5%, ±1%, ±0.5%, or even ±0.1% of the specified amount.

In one aspect, the present invention provides a new strategy for treatment of cancer by administering to a subject (e.g., a subject with cancer) a nanoparticle of this invention, which is engineered to selectively and directly targeting tumor cells in the subject. The nanoparticles deliver an active agent into the tumor cells to kill the cells and/or deposit a detectable marker to identify the cells as tumor cells.

Thus, in one embodiment, the present invention provides a nanoparticle comprising, consisting essentially of or consisting of: a) a polycation/polyalkylene glycol/glucose conjugate; and b) an active agent. In another embodiment, the present invention provides a nanoparticle comprising, consisting essentially of or consisting of: a) a core comprising an active agent; and b) a glucose/polyalkylene glycol conjugate surrounding the core of (a). In yet another embodiment, the present invention provides a nanoparticle comprising, consisting essentially of or consisting of: a) a core comprising an active agent; b) a glucose/polyalkylene glycol conjugate surrounding the core of (a); and c) a polycation. In a further embodiment, the present invention provides a nanoparticle comprising, consisting essentially of or consisting of a glucose/active agent complex. In yet another embodiment, the present invention provides a nanoparticle comprising, consisting essentially of or consisting of a) a polycation/alginate/glucose conjugate; and b) an active agent, as well as a nanoparticle comprising, consisting essentially of or consisting of a) a core comprising an active agent; and b) a glucose/alginate conjugate surrounding the core of (a). Additionally provided herein is a nanoparticle comprising, consisting essentially of or consisting of: a) a core comprising an active agent; b) a glucose/alginate conjugate surrounding the core of (a); and c) a polycation.

The nanoparticles of this invention can be present as a population of nanoparticles, thus the present invention provides a composition comprising a population of nanoparticles in a carrier. The nanoparticles of the population can be all of the same type or the population of nanoparticles can be made up of two or more different types of the nanoparticles as described herein in any combination and in any ratio.

The nanoparticles of the present invention are in a size range of about 20 nm to about 500 nm Thus, the nanoparticles can be 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 110 nm, 120 nm, 130 nm, 140 nm, 150 nm, 160 nm, 170 nm, 180 nm, 190 nm, 200 nm, 225 nm, 250 nm, 275 nm, 300 nm, 325 nm, 350 nm, 400 nm, 450 nm, 500 nm and the like or any combination thereof. It would be appreciated by one of skill in the art that in a composition comprising nanoparticles of this invention, the nanoparticles in the composition can vary in size, but will generally fall within the size range set forth herein.

In the nanoparticles of this invention, the glucose moiety of the conjugate can be present as glucose, as F-18-Fluordeoxyglucose (FDG), as radiolabeled glucose (e.g., [¹¹C]glucose, [¹⁴C]glucose), as a glucose derivative or any combination thereof. The term “glucose derivative” includes glucose molecules with a modified glucose chemical structure that is biocompatible and can be biochemically processed by the body (e.g., bound by the glucose transporter). Examples of glucose derivatives include, but are not limited to, dextraglucose (D-glucose), and 2-deoxyglucose (2-DG).

“Polyalkylene glycol” means straight or branched polyalkylene glycol polymers including, but not limited to, polyethylene glycol (PEG), polypropylene glycol (PPG), and polybutylene glycol (PBG), as well as co-polymers of PEG, PPG and PBG in any combination, and includes the monoalkylether of the polyalkylene glycol. Thus, in various embodiments of this invention, the polyalkylene glycol in the nanoparticles of this invention can be, but is not limited to, polyethylene glycol, polypropylene glycol, polybutylene glycol, and any combination thereof.

In certain embodiments, the polyalkylene glycol of the nanoparticle is polyethylene glycol or “PEG.” The term “PEG subunit” refers to a single polyethylene glycol unit, i.e., —(CH₂CH₂O)—.

In some embodiments, the polyalkylene glycol (e.g., PEG) can be non-polydispersed, monodispersed, substantially monodispersed, purely monodispersed, or substantially purely monodispersed.

“Monodispersed” is used to describe a mixture of compounds wherein about 100 percent of the compounds in the mixture have the same molecular weight.

“Substantially monodispersed” is used to describe a mixture of compounds wherein at least about 95 percent of the compounds in the mixture have the same molecular weight.

“Purely monodispersed” is used to describe a mixture of compounds wherein about 100 percent of the compounds in the mixture have the same molecular weight and have the same molecular structure. Thus, a purely monodispersed mixture is a monodispersed mixture, but a monodispersed mixture is not necessarily a purely monodispersed mixture.

“Substantially purely monodispersed” is used to describe a mixture of compounds wherein at least about 95 percent of the compounds in the mixture have the same molecular weight and have the same molecular structure. Thus, a substantially purely monodispersed mixture is a substantially monodispersed mixture, but a substantially monodispersed mixture is not necessarily a substantially purely monodispersed mixture.

In embodiments wherein the nanoparticle comprises, consists essentially of or consists of a) a polycation/alginate/glucose conjugate; and b) an active agent, the polycation, glucose and alginate can be attached to one another in the conjugate via covalent interactions. Furthermore, the polycation/alginate/glucose conjugate can be attached to the active agent via electrostatic forces.

In embodiments wherein the nanoparticle comprises, consists essentially of or consists of a) a core comprising an active agent; and b) a glucose/alginate conjugate surrounding the core of (a), the glucose and alginate can be attached to one another via covalent interactions. Furthermore, the glucose/alginate conjugate can be attached to the active agent via covalent interactions.

In some embodiments of the nanoparticles of this invention, a glucose/PAG conjugate (e.g., a glucose/PEG conjugate) can be present, surrounding a core comprising an active agent. In the glucose/PAG conjugate, the glucose and PAG can be attached, e.g., via chemically covalent bonding (e.g., covalent interactions). The PAG moiety of the glucose/PAG conjugate can be attached to the active agent of the core by covalent interactions.

Furthermore, in some embodiments of the nanoparticles of this invention, a polycation/PAG/glucose conjugate can be present, along with an active agent. In this conjugate, the glucose and PAG can be attached to one another as described above and the polycation (e.g., PEI) can be attached to the PAG (e.g., PEG) of the glucose/PAG conjugate, e.g., via chemically covalent bonding. The polycation of the conjugate can be attached to the active agent via electrostatic forces.

In nanoparticles of this invention in which a core comprising a polycation and an active agent is present, along with a glucose/PAG conjugate surrounding the core, the polycation can be attached to the active agent via electrostatic forces and the glucose/PAG conjugate can be attached to the polycation of the core via covalent bonding.

Additionally, in nanoparticles of this invention in which a core comprising a polycation and an active agent is present, along with a glucose/alginate conjugate surrounding the core, the polycation can be attached to the active agent via electrostatic forces and the glucose/alginate conjugate can be attached to the polycation of the core via covalent bonding.

In embodiments of this invention in which a polycation is present in the nanoparticle, the polycation can be, but is not limited to polyethyleneimine, polyethylenimine, poly(allylanion hydrochloride; PAH), putrescine, cadaverine, polylysine, poly-arginine, poly(trimethylenimine), poly(tetramethylenimine), polypropylenimine, aminoglycoside-polyamine, dideoxy-diamino-b-cyclodextrin, spermine, spermidine, cadaverine, poly(2-dimethylamino)ethyl methacrylate, poly(histidine), cationized gelatin, dendrimers, chitosan, and any combination thereof.

In certain embodiments of the nanoparticle of this invention, the polycation is polyethylenimine (PEI).

In various embodiments of this invention, the polycation (e.g., PEI) can be complexed with an active agent of this invention (e.g., a polynucleotide, an oligonucleotide, an anionic protein, an anionic drug, a polynucleotide or oligonucleotide covalently bonded to a peptide or protein, as well as any combination thereof) via physical electrostatic force (e.g., wherein the negative charges in the active agent(s) bind with the positive charges in the polycation. The active agent can be complexed with the polycation before and/or after the polycation is conjugated to GLU-PAG or GLU-alginate.

It is also envisioned that the various components of the nanoparticles of this invention can be attached to one another via crosslinking, e.g., with a crosslinking and/or catalyzing agent [e.g., 1-ethyl-(3-3-dimethylaminopropyl carbodiimide hydrochloride (EDC); N,N′-dicyclohexylcarbodiimide (DCC); N,N′-diisoproplycarbodiimide (DIC), genipin and any other crosslinking and/or catalyzing agent known in the art, in any combination]. In some embodiments, chemical crosslinking such as photocrosslinking can be employed, in which components with photocurable groups can be co-cross-linked with photocurable components of the nanoparticle.

In other embodiments, the components of the nanoparticle can be attached via a linking molecule. Examples of linking molecules of the invention include, but are not limited to, heparin and heparin sulphate. In embodiments in which heparin is used as a linking molecule, active agents can be used that bind to the heparin by electrostatic force or specific binding. For example, heparin has specific binding with TGF-β1, IL-10, HGF, FGF and others, as is well known in the art. Furthermore, heparin is negatively charged and can bind positively charged polycations via electrostatic forces. Additional linking molecules of this invention include heparin analogs and modified polysaccharides, e.g., as described in Frank et al. (J. Biol. Chem. 278(44):43229-43235 (2003)).

The active agent present in the nanoparticle of this invention can be, but is not limited to a polynucleotide, a fragment of a polynucleotide, an oligonucleotide, an antisense sequence, a ribozyme, a nucleotide sequence encoding a ribozyme, an interfering RNA (siRNA; shRNA, dsRNA), a microRNA (miRNA), a protein, a biologically active fragment of a protein, a peptide, a chemotherapeutic drug or agent, a cytotoxic agent, a radionuclide, a detectable marker, an imaging agent and any combination thereof. In some embodiments, the polynucleotide of this invention can be a plasmid or nucleic acid construct encoding a functional protein or other product that imparts a therapeutic effect. Nonlimiting examples of proteins encoded by the nucleic acid construct of this invention include p53, p53/p73OD chimera, interleukin-2 (IL-2), interleukin-4 (IL-4), interleukin-6 (IL-6), interleukin-7 (IL-7), interleukin-12 (IL-12), interleukin-15 (IL-15), interleukin-18 (IL-18), interleukin-21 (IL-21), interleukin-27 (IL-27), cesalin, CPT-11, interferon inducible protein-10 (IP-10), monokine induced by interferon gamma (Mig; CXCL9), tumor necrosis factor alpha (TNF-alpha), Fas ligand, PTEN, ARF (p14), Fas, CDK inhibitory protein (CIP) p15, CIP p27, CIP p21, beta-catenin, vascular endothelial growth factor receptor 2 (msFlk1), thymidine kinase, an anti-angiogenic factor, an apoptosis inducing factor (e.g., BIK, BNIP, NOXA PUMA, BAD, BAX, EGL-1, CED13), etc, as are known in the art or later identified.

In some embodiments in which the nanoparticle comprises a core comprising an active agent, the core can comprise, consist essentially of or consist of a polynucleotide/polycation (e.g., PEI) complex as the active agent.

In a particular embodiment, the polynucleotide is a plasmid or nucleic acid construct encoding p53. In this embodiment, the nanoparticle of this invention selectively and directly delivers the plasmid encoding p53 to a tumor cell. The p53 protein is produced in the transfected tumor cell, wherein it manifests its anti-tumor effect.

As used herein, “nucleic acid,” “nucleotide sequence,” and “polynucleotide” encompass both RNA and DNA, including cDNA, genomic DNA, mRNA, synthetic (e.g., chemically synthesized) DNA or RNA and chimeras of RNA and DNA. The term polynucleotide or nucleotide sequence refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid or nucleotide sequence of this invention.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An isolated cell refers to a cell that is separated from other components with which it is normally associated in its natural state. For example, an isolated cell can be a cell in culture medium (e.g., in vitro) and/or a cell in a pharmaceutically acceptable carrier of this invention. Thus, an isolated cell can be delivered to and/or introduced into a subject. In some embodiments, an isolated cell can be a cell that is removed from a subject and manipulated ex vivo and then returned to the subject.

The term “fragment,” as applied to a polynucleotide, will be understood to mean a nucleotide sequence of reduced length relative to a reference nucleic acid or nucleotide sequence and comprising, consisting essentially of and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

As used herein, a “functional” polypeptide or “functional protein” or “functional fragment” or “biologically active fragment” of a polypeptide is one that substantially retains at least one biological activity normally associated with that polypeptide (e.g., anti-tumor activity, protein binding, ligand or receptor binding). In particular embodiments, the “functional” polypeptide or protein or “functional fragment” substantially retains all of the activities possessed by the unmodified protein. By “substantially retains” biological activity, it is meant that the polypeptide or protein or fragment retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as protein binding and anti-tumor activity can be measured using assays that are well known in the art and as described herein.

By the terms “express,” “expressing” or “expression” with regard to a nucleic acid comprising a coding sequence, it is meant that the nucleic acid is transcribed, and optionally, translated. Typically, according to the present invention, expression of a coding sequence of the invention will result in production of the polypeptide or other product of the invention. The entire expressed polypeptide or fragment or other product can also function in intact cells without purification.

In some embodiments of this invention, the active agent can be an antisense nucleotide sequence or antisense oligonucleotide. The term “antisense nucleotide sequence” or “antisense oligonucleotide” as used herein, refers to a nucleotide sequence that is complementary to a specified target nucleotide sequence. Antisense oligonucleotides and nucleic acids that express the same can be made in accordance with conventional techniques. See, e.g., U.S. Pat. No. 5,023,243 to Tullis; U.S. Pat. No. 5,149,797 to Pederson et al. The antisense nucleotide sequence can be complementary to the entire target nucleotide sequence or a portion thereof of at least 10, 20, 40, 50, 75, 100, 150, 200, 300, or 500 contiguous bases and will reduce the level of production of the protein or product encoded by the target nucleotide sequence.

Those skilled in the art will appreciate that it is not necessary that the antisense nucleotide sequence be fully complementary to the target nucleotide sequence as long as the degree of sequence similarity is sufficient for the antisense nucleotide sequence to hybridize to its target and reduce production of the encoded polypeptide or product. As is known in the art, a higher degree of sequence similarity is generally required for short antisense nucleotide sequences, whereas a greater degree of mismatched bases will be tolerated by longer antisense nucleotide sequences.

For example, hybridization of such antisense nucleotide sequences can be carried out under conditions of reduced stringency, medium stringency or even high stringency (e.g., conditions represented by a wash stringency of 35-40% formamide with 5×Denhardt's solution, 0.5% SDS and 1×SSPE at 37° C.; conditions represented by a wash stringency of 40-45% formamide with 5×Denhardt's solution, 0.5% SDS, and 1×SSPE at 42° C.; and/or conditions represented by a wash stringency of 50% formamide with 5×Denhardt's solution, 0.5% SDS and 1×SSPE at 42° C., respectively) with respect to their target nucleotide sequences. See, e.g., Sambrook et al., Molecular Cloning: A Laboratory Manual 2nd Ed. (Cold Spring Harbor, N.Y., 1989). Thus, in some embodiments, an antisense nucleotide sequence of this invention can have at least about 70%, 80%, 90%, 95%, 97%, 98% or higher sequence similarity with the complement of the target coding sequence and will reduce the level of polypeptide production.

The length of the antisense nucleotide sequence (i.e., the number of nucleotides therein) is not critical as long as it binds selectively to the intended location and reduces transcription and/or translation of the target sequence, and can be determined in accordance with routine procedures. In general, the antisense nucleotide sequence will be from about eight, ten or twelve nucleotides in length up to about 20, 30, 50, 75, 100, 150, 200, 250, 300, 350, 400, 450 or 500 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

The antisense nucleotide sequences of the invention further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., Nucleic Acids Res. 17:9193 (1989); Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989); Walder and Walder, Proc. Natl. Acad. Sci. USA 85:5011 (1988); incorporated by reference herein in their entireties for their teaching of methods of making antisense molecules, including those containing modified nucleotide bases).

In various embodiments of this invention, the active agent can be an interfering RNA, such as a small interfering RNA (siRNA), a short hairpin RNA (shRNA), a double stranded RNA (dsRNA) or a microRNA (miRNA). These interfering RNAs can be directed against target nucleotide sequences within a tumor cell, resulting in modulation of the expression of nucleotide sequences and subsequent modulation of the production of the protein or product encoded by the target nucleotide sequence. Nonlimiting examples of a nucleotide sequence to be targeted in a tumor cell or cancer cell using an interfering RNA approach include Bel-2, Bel-XL, Akt, HIF-a, MMP, Ras and MDM2.

siRNA is a mechanism of post-transcriptional gene silencing in which double-stranded RNA (dsRNA) corresponding to a target coding sequence is introduced into a cell or an organism, resulting in degradation of the corresponding mRNA. The mechanism by which siRNA achieves gene silencing has been reviewed in Sharp et al., Genes Dev. 15:485 (2001); and Hammond et al., Nature Rev. Gen. 2:110 (2001)). The siRNA effect persists for multiple cell divisions before gene expression is regained. siRNA has proven successful in human cells, including human embryonic kidney and HeLa cells (see, e.g., Elbashir et al., Nature 411:494 (2001)). In one embodiment, silencing can be induced in mammalian cells by enforcing endogenous expression of RNA hairpins (shRNA) (see Paddison et al., Proc. Natl. Acad. Sci. USA 99:1443 (2002)). In another embodiment, transfection of small (21-23 nt) dsRNA specifically inhibits nucleic acid expression (reviewed in Caplen, Trends Biotechnol. 20:49 (2002)).

siRNA technology utilizes standard molecular biology methods. dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in siRNA are available commercially, e.g., from New England Biolabs, Inc. Methods of transfection of dsRNA or plasmids engineered to make dsRNA are routine in the art.

MicroRNAs (miRNAs), which are single stranded RNA molecules of about 21-23 nucleotides in length, can be used in a similar fashion to siRNA to modulate gene expression (see U.S. Pat. No. 7,217,807).

Silencing effects similar to those produced by siRNA have been reported in mammalian cells with transfection of a mRNA-cDNA hybrid construct (Lin et al., Biochem. Biophys. Res. Commun. 281:639 (2001)), providing yet another strategy for silencing a coding sequence of interest.

The active agent of this invention can also, in some embodiments, be a ribozyme, as well as a nucleic acid encoding a ribozyme. Ribozymes are RNA-protein complexes that cleave nucleic acids in a site-specific fashion. Ribozymes have specific catalytic domains that possess endonuclease activity (Kim et al., Proc. Natl. Acad. Sci. USA 84:8788 (1987); Gerlach et al., Nature 328:802 (1987); Forster and Symons, Cell 49:211 (1987)). For example, a large number of ribozymes accelerate phosphoester transfer reactions with a high degree of specificity, often cleaving only one of several phosphoesters in an oligonucleotide substrate (Michel and Westhof, J. Mol. Biol. 216:585 (1990); Reinhold-Hurek and Shub, Nature 357:173 (1992)). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, Nature 338:217 (1989)). For example, U.S. Pat. No. 5,354,855 states that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., Proc. Natl. Acad. Sci. USA 88:10591 (1991); Sarver et al., Science 247:1222 (1990); Sioud et al., J. Mol. Biol. 223:831 (1992)).

In some embodiments, the active agent of this invention can be a chemotherapeutic drug or agent. Nonlimiting examples of a chemotherapeutic drug or agent include 1) vinca alkaloids (e.g., vinblastine, vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C); 4) enzymes (e.g., L-asparaginase); 5) biological response modifiers (e.g., interferon-alpha); 6) platinum coordinating complexes (e.g., cisplatin and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide). Additional examples of a chemotherapeutic of this invention include methotrexate, epirubicin, fluorouracil, verapamil, cyclophosphamide, cytosine arabinoside, aminopterin, bleomycin, democolcine, etoposide, mithramycin, chlorambucil, melphalan, daunorubicin, doxorubicin, tamoxifen, paclitaxel, camptothecin, matrix metalloproteinase (MMP) inhibitors (e.g., Marimastat, Trocade, doxycycline, minocycline and as described in U.S. Pat. No. 5,872,152, incorporated by reference herein), and cytarabine.

In further embodiments of this invention, the active agent can be a radionuclide. “Radionuclide” as described herein may be any radionuclide suitable for delivering a therapeutic dosage of radiation to a tumor or cancer cell, including but not limited to ²²⁷Ac, ²¹¹At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²P, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵Ag, ⁸⁹Sr, ³⁵S, ¹⁷⁷Ta, ¹¹⁷mSn, ¹²¹Su, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, and ²¹²Pb.

In yet further embodiments of this invention, the active agent can be or can include a detectable marker or label. Nonlimiting examples of detectable markers or labels include radionuclides (³⁵S, ¹²⁵I, ¹³¹I, ⁹⁹Te, ⁶⁴CU, etc), enzymes, fluorescence agents, chemiluminescence agents and chromogenic agents, as well as substrates, cofactors, inhibitors, magnetic particles and the like as are well known in the art.

An active agent of this invention can also be a cytotoxic agent. “Cytotoxic agent” as used herein includes but is not limited to ricin (e.g., ricin A chain), aclacinomycin, diphtheria toxin, Monensin, Verrucarin A, Abrin, vinca alkaloids, tricothecenes and Pseudomonas exotoxin A.

In addition, an active agent of this invention can be an imaging agent. For example, fluorescein isothiocyanate (FITC) can be the imaging agent for fluorescence imaging, superparamagnetic iron oxide can be the imaging agent for magnetic resonance imaging (MRI) and/or radionuclides can be the imaging agent for radiographic imaging.

It is to be understood that any of the elements of the nanoparticles of this invention can be present in the various embodiments of the nanoparticles of this invention as described above and any such elements can also be absent from or excluded from the nanoparticles of this invention and such exclusion or absence thereof can be defined as a negative limitation of this invention (e.g., excluded from a described embodiment by negative proviso).

As a further aspect, the invention provides pharmaceutical formulations and methods of administering the same to achieve a therapeutic effect and/or for diagnosis, as described herein. The pharmaceutical formulation may comprise any of the nanoparticles described herein in a pharmaceutically acceptable carrier.

A “pharmaceutically acceptable carrier” is a component such as a salt, carrier, excipient or diluent of a composition that is (i) compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition.

Non-limiting examples of pharmaceutically acceptable components include, without limitation, any of the standard pharmaceutical carriers such as phosphate buffered saline solutions, water, emulsions such as oil/water emulsion, microemulsions and various types of wetting agents. In particular, it is intended that a pharmaceutically acceptable carrier be a sterile carrier that is formulated for administration to or delivery into a subject of this invention. Furthermore, a pharmaceutically acceptable carrier is any carrier molecule approved by a regulatory agency of the federal or a state government or listed in the U.S. Pharmacopeia or other generally recognized pharmacopeia for use in animals, and more particularly in humans. Such carrier can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Examples of suitable pharmaceutical carriers are well known to those skilled in the art (See, for example, Remington: The Science and Practice of Pharmacy, 21^{st} Edition (2005), Lippincott Williams & Wilkins, Philadelphia, Pa.).

Further, in preparing such pharmaceutical compositions comprising the active ingredient or ingredients in admixture with components necessary for the formulation of the compositions, other conventional pharmacologically acceptable additives may be incorporated, including, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, dextrose, lactose, glucose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

The formulations of the invention can optionally comprise medicinal agents, pharmaceutical agents, carriers, adjuvants, dispersing agents, diluents, and the like.

The nanoparticles of the invention can be formulated for administration in a pharmaceutical carrier in accordance with known techniques. See, e.g., Remington: The Science and Practice of Pharmacy, 21^{st} Edition (2005), Lippincott Williams & Wilkins, Philadelphia, Pa.). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo, comprising administering to a subject a pharmaceutical composition comprising a nanoparticle of the invention in a pharmaceutically acceptable carrier, wherein the pharmaceutical composition is administered in a therapeutically effective amount. Administration of the compositions of the present invention to a human subject or an animal in need thereof can be by any means known in the art for administering such compositions.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, intraocular, intravisceral, intraretinal, transdermal, intraarticular, intrathecal, and inhalation administration, administration to the liver by intraportal delivery, as well as direct organ injection (e.g., into the liver, into the brain for delivery to the central nervous system, into the pancreas, or into a tumor or the tissue surrounding a tumor). The most suitable route in any given case will depend on the nature and severity of the condition being treated and on the nature of the particular compound which is being used.

For injection, the carrier will typically be a liquid, such as sterile pyrogen-free water, pyrogen-free phosphate-buffered saline solution, bacteriostatic water, or Cremophor EL[R](BASF, Parsippany, N.J.). For other methods of administration, the carrier can be either solid or liquid.

For oral administration, the composition can be administered in solid dosage forms, such as capsules, tablets, and powders, or in liquid dosage forms, such as elixirs, syrups, and suspensions. Compositions can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the composition in a flavored base, usually sucrose and acacia or tragacanth; and pastilles comprising the composition in an inert base such as gelatin and glycerin or sucrose and acacia.

Formulations of the present invention suitable for parenteral administration comprise sterile aqueous and non-aqueous injection solutions of the composition, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a nanoparticle of the invention, in a unit dosage form in a sealed container. The composition is provided in the form of a lyophilizate which is capable of being reconstituted with a suitable pharmaceutically acceptable carrier to form a liquid composition suitable for injection thereof into a subject. When the composition is substantially water-insoluble, a sufficient amount of emulsifying agent which is pharmaceutically acceptable can be employed in sufficient quantity to emulsify the composition in an aqueous carrier. One such useful emulsifying agent is phosphatidyl choline.

Formulations suitable for rectal administration are preferably presented as unit dose suppositories. These can be prepared by admixing the composition with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the composition. Suitable formulations comprise citrate or bis\tris buffer (pH 6) or ethanol/water.

The compound can alternatively be formulated for nasal administration or otherwise administered to the lungs of a subject by any suitable means, e.g., administered by an aerosol suspension of respirable particles comprising the nanoparticles, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the nanoparticles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the nanoparticles can likewise be produced with any solid particulate medicament aerosol generator, by techniques known in the pharmaceutical art.

Alternatively, one can administer the nanoparticles of this invention locally (e.g., directly into and/or proximal to a tumor) rather than systemically, for example, in a depot or sustained-release formulation.

The nanoparticles of the present invention can optionally be delivered in conjunction with other therapeutic agents and/or treatments. The additional therapeutic agents and/or treatments can be delivered before, after and/or concurrently with the nanoparticles of the invention. As used herein, the word “concurrently” means sufficiently close in time to produce a combined effect (that is, concurrently can be simultaneously, or it can be two or more events occurring within a short time period before or after each other). In one embodiment, the nanoparticles of the invention are administered in conjunction with anti-cancer agents (e.g., chemotherapeutic drugs or agents as described herein) and/or with anti-angiogenesis agents, nonlimiting examples of which include antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide TNF alpha conjugate, cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736, AMG-706, AZD2171, sorafenib tosylate, BMS-582664, CHIR-265, pazopanib, PI-88, vatalanib, everolimus, suramin, sunitinib malate, XL184, ZD6474, ATN-161, cilenigtide, and celecoxib. Furthermore, a subject of this invention can be receiving radiation treatment and/or surgical treatment in combination with the delivery of nanoparticles and can also be receiving chemotherapeutic drugs, cytokines, hormones and/or anti-angiogenic agents, as is known in the art.

In particular embodiments, the nanoparticles are administered to the subject in a therapeutically effective amount, as that term is defined herein. Dosages of pharmaceutically active compounds can be determined by methods known in the art, see, e.g., Remington's Pharmaceutical Sciences (Maack Publishing Co., Easton, Pa.). The therapeutically effective dosage of any specific nanoparticle composition will vary somewhat from composition to composition, and subject to subject, and will depend upon the condition of the subject, the composition of the particular nanoparticle and/or the route and/or frequency of delivery.

For administration/delivery of nanoparticles comprising a nucleic acid, a dosage range from about 10⁹ to about 10¹² nanoparticles/cm² (e.g., 10⁹/cm², 10¹⁰/cm², 10¹¹/cm², 10¹²/cm² nanoparticles) can be used. The number of nanoparticles in a given volume of carrier or vehicle (e.g., phosphate buffered saline (PBS); hydrogel, etc.) is determined via spectrophotometric analysis or via any other method known in the art for quantifying particles.

For administration/delivery of a nanoparticle comprising a therapeutic drug, a dosage range can be from about 5 mg/m² to about 200 mg/m² of the nanoparticles or more particularly, from about 10 mg/m² to about 100 mg/m² of the nanoparticles, (e.g., 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 5 mg/m², 19 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 125 mg/m², 150 mg/m², 175 mg/m², 200 mg/m² of the nanoparticles), dependent on which drug is used, as would be determined according to methods standard in the art.

Exemplary dosage ranges for administration/delivery of the nanoparticle using mg/kg include from about 2 mg/kg to about 20 mg/kg of nanoparticles, as well as a range from about mg/kg to about 10 mg/kg of nanoparticles (e.g., 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of nanoparticles).

For administration/delivery of a p53 chimera of this invention (e.g., p53/p73OD), an exemplary dosage range can be from about 1.0 mg DNA/kg to about 10.0 mg DNA/kg (e.g., 1.0, 1.5, 2.0. 2.5, 3.0 3.5, 4.0. 4.5, 5.0, 5.5, 6.0, 6.5, 7.0, 7.5, 8.0, 8.5, 9.0, 9.5 or 10.0 mg DNA/kg).

In particular embodiments of the invention, more than one administration (e.g., two, three, four, or more administrations) can be employed over a variety of time intervals (e.g., hourly, daily, weekly, monthly, annually etc.) to achieve therapeutic effects and/or for diagnostic purposes.

Thus, in particular embodiments, the present invention provides various methods employing the nanoparticles of this invention. In particular, provided herein is a method of delivering a nanoparticle to a cell, comprising contacting the cell with a nanoparticle of this invention under conditions whereby the nanoparticle binds a glucose transporter at the cell surface and is internalized by the cell. The cell can be in vivo (i.e., a cell in a subject) or the cell can be in vitro or ex vivo. Furthermore, a cell of this invention can be a cancer cell, a precancerous cell or a tumor cell.

Further provided herein is a method of delivering an active agent to a tumor cell in a subject in need thereof, comprising delivering a nanoparticle of this invention to the subject, whereby the nanoparticle binds a glucose transporter at the tumor cell surface and is internalized by the tumor cell, thereby delivering the active agent to the tumor cell.

Additionally, the present invention provides a method of decreasing the size of a tumor in a subject in need thereof, comprising delivering an effective amount of a nanoparticle of this invention to the subject, thereby decreasing the size of the tumor in the subject.

Also provided herein is a method of inducing tumor regression in a subject in need thereof, comprising delivering an effective amount of a nanoparticle of this invention to the subject, thereby inducing tumor regression in the subject. Tumor regression can be determined, for example, in cancers that are not solid tumor cancers, such as leukemia and other “liquid cancers” as are well known in the art.

The present invention further provides a method of treating cancer in a subject (e.g., a subject in need thereof), comprising delivering an effective amount of a nanoparticle of this invention to the subject, thereby treating cancer in the subject. The present invention also provides a method of treating a hyperproliferative disorder (e.g., wherein hyperproliferative cells overexpress glucose transporters on the cell surface) in a subject (e.g., a subject in need thereof), comprising delivering an effective amount of a nanoparticle of this invention to the subject, thereby treating the hyperproliferative disorder in the subject.

The term “cancer,” a used herein, refers to any benign or malignant abnormal growth of cells. The cancer of this invention can be a primary cancer and/or a metastatic cancer. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. Further provided herein is any cancer that is associated with a mutant p53 phenotype, as is known in the art.

The term “subject” as used herein includes any subject in whom the methods of this invention can be carried out. Subjects to whom the nanoparticles of this invention can be delivered according to the methods of the present invention include both human subjects for medical purposes (e.g., therapeutic and/or diagnostic) and animal subjects for veterinary and drug screening and development purposes. In some embodiments, the subject can be an avian subject or a mammalian subject (e.g., dog, cat, horse, cow, sheep, goat, primate, rat, mouse, lagomorphs, rabbits, guinea pigs, hamsters, etc.), and in particular embodiments is a human subject (including both male and female subjects, and including neonatal, infant, juvenile, adolescent, adult, and geriatric subjects, further including pregnant subjects). In other embodiments, the subject is an animal model of cancer, tumor growth and/or other hyperproliferative disorders.

A subject “in need thereof” includes, but is not limited to, a subject diagnosed with cancer or other proliferative disorder, a subject suspected of having cancer, a subject having a tumor, a subject suspected of having a tumor, a subject at increased risk of having cancer or other proliferative disorder, a subject likely to have cancer, a subject likely to have a tumor etc. Such a subject is one who is therefore in need of and/or would benefit from and/or desires having the nanoparticles of this invention administered or delivered thereto for therapeutic and/or diagnostic purposes.

The term “therapeutically effective amount” or “effective amount,” as used herein, refers to that amount of a nanoparticle of this invention and/or a composition comprising a nanoparticle of this invention that imparts a modulating effect, which, for example, can be a beneficial effect, to a subject afflicted with a condition (e.g., a disorder, disease, syndrome, illness, injury, traumatic and/or surgical wound), including improvement in the condition of the subject (e.g., in one or more symptoms), delay or reduction in the progression of the condition, prevention or delay of the onset of the condition, and/or change in clinical parameters, status or classification of a disease or illness, etc., as would be well known in the art.

For example, a therapeutically effective amount or effective amount can refer to the amount of a nanoparticle or composition of this invention that improves a condition (e.g., treats cancer and/or reduces tumor size and/or induces tumor regression) in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat,” “treating,” “treatment” or “healing” refers to any type of action that imparts a modulating effect, which, for example, can be a beneficial effect, to a subject afflicted with a condition (e.g., disorder, disease, syndrome, illness, traumatic or surgical wound, injury, etc.).

By the terms “treat,” “treating,” “healing” or “treatment of” (or grammatically equivalent terms), it is also meant that the severity of the subject's condition is reduced or at least partially improved or ameliorated and/or that some alleviation, mitigation or decrease in at least one clinical symptom is achieved and/or there is a delay in the progression of the condition and/or delay of the onset of a disease or disorder.

By “prevent,” “preventing” or “prevention” is meant to avoid or eliminate the development and/or manifestation of a pathological state and/or disease condition or status in a subject.

In the methods of this invention in which tumor size is reduced, the size of the tumor can be reduced (e.g., reduced by 10%, 20%, 30%, 40%, 50%, 60%, 70%, 80%, 90%, 95% or 100%) as compared to the size of the tumor without treatment with the nanoparticles and/or compositions of this invention. Efficacy of treatment can also be determined by monitoring a decrease in the growth rate of a tumor or induction of tumor regression (e.g., growth rate reduced or regression induced, by about 10%, 20%, 30%, 40%, 50%, 60%, 70%, 80%, 90%, 95% or 100%) as compared to the growth rate of the tumor without treatment with the nanoparticles and/or compositions of this invention. Other parameters that can be measured to determine efficacy are the rate/degree of apoptosis and/or senescence of the tumor cells in response to treatment with the nanoparticles and/or compositions of this invention.

The nanoparticles of the present invention are delivered to the subject via a variety of methods, including, but not limited to, oral delivery, intravenous delivery, subcutaneous delivery, injection, surgical implantation, delivery into a body cavity, topical application, and any combination thereof.

The present invention further provides methods of diagnosing cancer in a subject. In one embodiment, an in vitro method of diagnosing cancer in a subject is provided, comprising: a) contacting a nanoparticle of this invention with cells from the subject; b) measuring the rate and/or amount and/or selectivity of internalization of the nanoparticles into the cells of the subject; and c) comparing the rate and/or amount and/or selectivity of internalization of the nanoparticles into the cells of the subject with the rate and/or amount and/or selectivity of internalization of the nanoparticles into cells of a control subject and/or in control cells of the subject being diagnosed, whereby an increase in the amount and/or rate of internalization and/or a demonstrated selectivity of the nanoparticles into the cells of the subject as compared with the cells of the control subject and/or with the control cells of the test subject is diagnostic of cancer in the subject. Methods of measuring the rate and/or amount and/or selectivity of internalization of the nanoparticles of this invention into cells are as described in the examples set forth herein and as are well known in the art. Nonlimiting examples include detection of a marker (e.g., GFP) and measurement of expression of a nucleotide sequence carried by the nanoparticle to produce a product (e.g., beta galactosidase).

Also provided herein is an in vivo method of diagnosing cancer in a subject, comprising: a) delivering a nanoparticle of this invention to the subject, wherein the nanoparticle comprises an imaging agent; b) detecting a signal from the imaging agent in the subject; and c) comparing the signal from the imaging agent in the subject with the signal from the same imaging agent in a control subject or in a control tissue from the subject being diagnosed, whereby an alteration (e.g., accumulation of signal in a particular organ, cell type, location, etc.) in the signal from the subject as compared with the signal from the control subject or the control tissue is diagnostic of cancer in the subject. Methods of detecting a signal from the imaging agents of this invention when said imaging agents are present within a subject are well known in the art.

It is further contemplated that the methods of this invention can be employed to monitor tumor dynamics (e.g., reduction in tumor size over time following delivery of the nanoparticles of this invention to a subject) and to identify effective treatments. The nanoparticles of this invention can be engineered to simultaneously impart a therapeutic effect and provide a detectable signal for such monitoring methods.

Embodiments of the present invention further include a kit comprising one or more of the nanoparticles and/or compositions described herein and optionally instructions for use and/or administration. It would be well understood by one of skill in the art that the kits of this invention can comprise one or more containers and/or receptacles to hold the reagents of the kit, along with appropriate reagents and directions for using the kit, as would be well known in the art. Each of these components of the kit can be combined in the same container and/or provided in separate containers.

In additional aspects, the present invention provides a derivative of p53, which is a p53 chimera comprising a p73OD. Also provided herein is a composition comprising, consisting essentially of or consisting of a p53 chimera comprising a p73OD, in a pharmaceutically acceptable carrier.

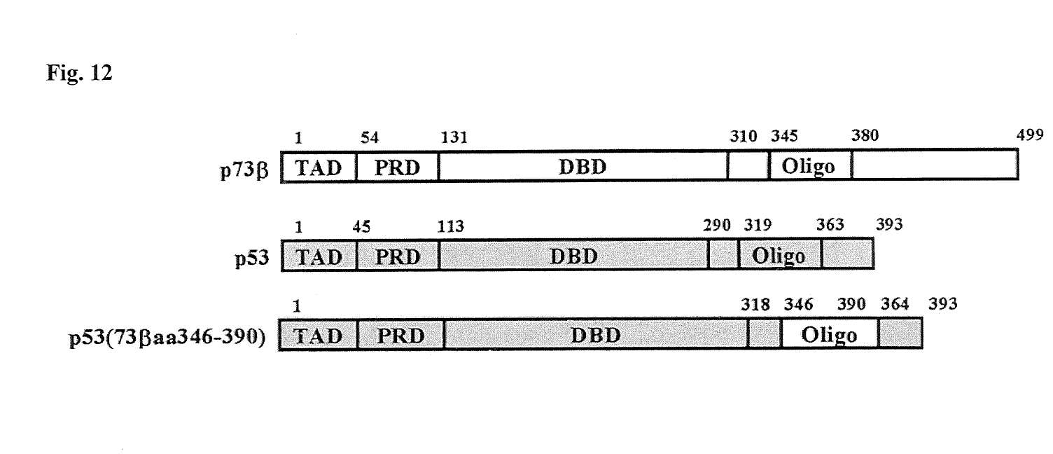

A p53 chimera comprising a p73 oligimerization domain (OD), also known as p53/p73OD, is a human p53 protein having 393 amino acids, in which amino acids 318 to 364 are replaced with amino acids 346-390 of p73 (the oligomerization domain) [28]. The amino acid sequence of each of wild type p53 (e.g., Gene ID 7157 in PubMed), wild type p73 (e.g., Gene ID 7161 in PubMed) and the chimera p53/p73OD is shown below, with the substituted sequences in bold. Schematics of these wild type and chimera (described in the figure as p53(73βaa346-390) are shown in FIG. 12, with the various domains identified. It would be readily understood by one of ordinary skill in the art that various substitutions to the amino acid sequence of the p53 chimera could be made without altering the function of the p53 chimera of this invention. The production of such variant amino acid sequences and assays to test for functionality of the resulting variant p53 chimera are well within the skill of the ordinary artisan. Thus, such variants of the p53 chimera sequence provided herein are included within the present invention.

p73 (499 amino acids; GenBank Database Accession No. CAA72219) Amino acids 346-390 are bolded.

maqstatspd ggttfehlws slepdstyfd lpqssrgnne
vvggtdssmd vfhlegmtts vmaqfnllss tmdqmssraa
saspytpeha asvpthspya qpsstfdtms papvipsntd
ypgphhfevt fqqsstaksa twtyspllkk lycqiaktcp
iqikvstppp pgtairampv ykkaehvtdv vkrcpnhelg
rdfnegqsap ashlirvegn nlsqyvddpv tgrqsvvvpy
eppqvgteft tilynfmcns scvggmnrrp iliiitlemr
dgqvlgrrsf egricacpgr drkadedhyr eqqalnessa
kngaaskraf kqsppavpal gagvkKRRHG DEDTYYLQVR
GRENFEILMK LKESLELMEL VPQPLVDSYR qqqqllqrps
hlqppsygpv lspmnkvhgg mnklpsvnql vgqppphssa
atpnlgpvgp gmlnnhghav pangemsssh saqsmvsgsh
ctppppyhad pslvrtwgp

p53 (393 amino acids; GenBank®Database Accession No. NP_{—}00537). Amino acids 318-364 are bolded.

meepqsdpsv epplsqetfs dlwkllpenn vlsplpsqam
ddlmlspddi eqwftedpgp deaprmpeaa ppvapapaap
tpaapapaps wplsssvpsq ktyqgsygfr lgflhsgtak
svtctyspal nkmfcqlakt cpvqlwvdst pppgtrvram
aiykqsqhmt evvrrcphhe rcsdsdglap pqhlirvegn
lrveylddrn tfrhsvvvpy eppevgsdct tihynymcns
scmggmnrrp iltiitleds sgnllgrnsf evrvcacpgr
drrteeenlr kkgephhelp pgstkralpn ntssspqpkk
kpldgeyftl qirgrerfem frelnealel kdaqagkepg
gsrahsshlk skkgqstsrh kklmfktegp dsd

p53/p73OD. Amino acids 318-364 of p53 (393 amino acid sequence) are substituted with amino acids 346-390 of p73 (499 amino acid sequence).

meepqsdpsv epplsqetfs dlwkllpenn vlsplpsqam
ddlmlspddi eqwftedpgp deaprmpeaa ppvapapaap
tpaapapaps wplsssvpsq ktyqgsygfr lgflhsgtak
svtctyspal nkmfcqlakt cpvqlwvdst pppgtrvram
aiykqsqhmt evvrrcphhe rcsdsdglap pqhlirvegn
lrveylddrn tfrhsvvvpy eppevgsdct tihynymcns
scmggmnrrp iltiitleds sgnllgrnsf evrvcacpgr
drrteeenlr kkgephhelp pgstkralpn ntssspqK
RRHGDEDTYY LQVRGRENFE ILMKLKESLE LMELVPQPLV
DSYRhss hlkskkgqst srhkklmfkt egpdsd

Certain aspects of this invention are based on the unexpected discovery that the p53 chimera of this invention does not bind mutant p53 in tumor cells and thus can effectively restore p53 function in the presence of a high level of p53 mutants. Accordingly, the p53 chimera of this invention can be used as a therapeutic to treat cancer and/or to reduce tumor size. Therefore in one aspect, the present invention provides a method of delivering a p53 chimera comprising a p73OD (e.g., p53/p73OD) to a cell, comprising contacting the cell with the p53 chimera under conditions whereby the p53 chimera is internalized by the cell. In various embodiments of this method, the cell can be in vivo, in vitro or both. Further provided herein is a method of decreasing the size of a tumor in a subject in need thereof, comprising, consisting essentially of or consisting of introducing an effective amount of a p53 chimera comprising a p73 OD into tumor cells of the subject, thereby decreasing the size of the tumor in the subject.

The present invention additionally provides a method of treating cancer in a subject in need thereof, comprising, consisting essentially of or consisting of delivering an effective amount of a p53 chimera comprising a p73 OD to the subject, thereby treating cancer in the subject.

The p53 chimera of this invention can be included as part of the nanoparticle of this invention, as described above. However, it is understood that delivery of the p53 chimera to a subject is not limited to introduction into cells of a subject as a part of the nanoparticle of this invention. Specifically, such delivery can include introducing the p53 chimera of this invention into a cell of a subject of this invention by any vehicle and/or mechanism known for introducing a nucleic acid molecule into a cell. Numerous protocols for the delivery of a nucleic acid molecule into a subject and into a cell of a subject are well known in the art and are encompassed within the methods of this invention. Nonlimiting examples include plasmids, expression vectors, viral vectors (e.g., lentivirus, retrovirus, adenovirus, adeno-associated virus (AAV), alphavirus, poxvirus, etc.), liposomes, naked nucleic acid molecules, etc., as are well known. In particular embodiments of this invention, the p53 chimera can be introduced into cells via an adenovirus-mediated gene transfer system, which is well known in the art.

In the methods of this invention employing a nanoparticle and/or chimera of this invention, such methods can further comprise, consist essentially of or consist of the step of administering to the subject a chemotherapeutic agent, an anti-angiogenic agent, a cytokine, a hormone, a radiation treatment, a surgical treatment or any combination thereof, according to protocols well known in the art. Such a further step can be carried out before, after and/or simultaneously with the delivery of the p53 chimera to the subject.

Furthermore, in the methods of this invention employing nanoparticles and/or a p53 chimera, a further step can be included, comprising, consisting essentially of or consisting of administering to the subject an inhibitor of protein kinase, an inhibitor of histone deacetylase (HDAC), an inhibitor of methyltransferase, and any combination thereof. Further provided herein is a kit comprising a composition comprising the chimera of this invention in a pharmaceutically acceptable carrier.

The present invention is more particularly described in the following examples that are intended as illustrative only since numerous modifications and variations therein will be apparent to those skilled in the art.

### EXAMPLES

### Example 1

### A Cancer Cell Specific Nanoparticle Delivery System

In the present invention, a previously undescribed type of cancer cell-targeting nanoparticle was developed by exploiting the Warburg effect, i.e., the phenomenon whereby cancer cells take up more glucose than healthy cells. Specifically, a glucose-conjugated PEG-linked polyethyleneimine (PEI) conjugate was synthesized (GLU-PEG-PEI). Using this system to deliver an EGFP or beta-galactosidase (β-Gal) expression plasmid in cell culture studies, a considerablly greater efficiency of delivery was obtained in the cancer cell lines, PC3 or MDA-MB-231, as compared with the non-cancer cell lines, RWPE or MCF-10A cells. The specificity of delivery is glucose transporter specific, as evidenced by the finding that expression of EGFP or β-Gal was markedly inhibited by addition of an excess amount of glucose. Further analysis indicated that the GLU-PEG-PEI/DNA complex enters cells via endocytosis-mediated internalization of the nanoparticle/glucose transporter complex. Collectively, these results provide proof-of-principle for the potential of the delivery system in cancer therapeutics.

The effectiveness of anticancer therapeutics relies on the ability to reduce and eliminate cancer cells without damaging healthy tissues. Thus, a strategy of preferentially targeting cancer cells is essential in the success of cancer therapeutics. Encapsulation of therapeutic agents inside a nanotechnology-based drug delivery system has been a widely applied formulation strategy. There are a variety of nanoparticle systems currently being explored to enhance delivery of therapeutic drugs to cancer cells, including various liposomes, polymeric micelles, dendrimers, protein nanoparticles, viral nanoparticles, carbon nanotubes and cationic polymers [24]. Accumulation of nanoparticles in tumor tissue is a passive process that depends on a long circulating half-life to facilitate extravasation of nanoparticles through the tumor microvasculature and accumulation of therapeutic products in the tumor tissue.

One critical strategy of prolonging circulation time is to evade uptake by tissue macrophages and to resist removal by reticuloendothelial systems. For this purpose, nanoparticles have been coated with inert and biocompatible polymers to create a stealth surface. For instance, incorporation of a hydrophilic polymer, such as poly-ethylene glycol (PEG) or alginate, to the surface of the nanoparticle has been widely used for increasing circulation time [25]. Once nanoparticles extravasate out of the vasculature into tumor tissues, their uptake by tumor cells is facilitated by active targeting, which can result in higher intracellular concentration of therapeutic products and subsequently increased therapeutic efficacy. Active targeting involves the use of perpherally conjugated targeting ligands for enhanced delivery. Cell proliferation markers represent a class of important targets for cancer therapeutics because many of these markers are highly expressed on certain cancer cells. The most established cancer cell targets utilized by actively targeting nanoparticles include antibodies against human epidermal receptors, transferring receptors, and folate receptors. Although antibody targeting is thought of as a promising strategy, it has several drawbacks, including the large hydrodynamic size, which limits both intratumoral uptake and homogeneous distribution in the tumor, thus adversely affecting pharmacokinetic properties. New methods of targeting have been recently developed. Among them, nucleic acid ligands (aptamers) have gained substantial interest. Aptamers are DNA or RNA oligonucleotides that, via intramolecular interactions, fold into unique tertiary conformations that bind to target antigens with high affinity and specificity. In comparison with probes currently available for biomarker recognition, aptamers possess high specificity, low molecular weight, easy and reproducible production, versatility in application, and easy manipulation [24]. While the use of aptamers represents a significant advance of targeted nanoparticle delivery, the identification of cancer cell specific aptamers remains challenging and has been the bottleneck of such an approach.

The present invention is based on the development of a novel form of cancer cell-targeting nanoparticle by exploiting the Warburg effect, i.e., cancer cells generally take up more glucose than healthy cells. Specifically, in the present invention, glucose-conjugated PEG-linked (GLU-PEG) nanoparticles were synthesized that can utilize highly overexpressed glucose transporters to preferentially enter cancer cells. Recognizing the emerging therapeutic potential of new classes of bioactive macromolecules, such as antisense RNA, RNAi, and miRNA, the use of these GLU-PEG nanoparticles was analyzed for intracellular delivery of expression plasmids. Polyethylenimine (PEI) was used to synthesize GLU-PEG-PEI for delivery of a GFP-expressing or β-galactosidase-expressing plasmid into cells.

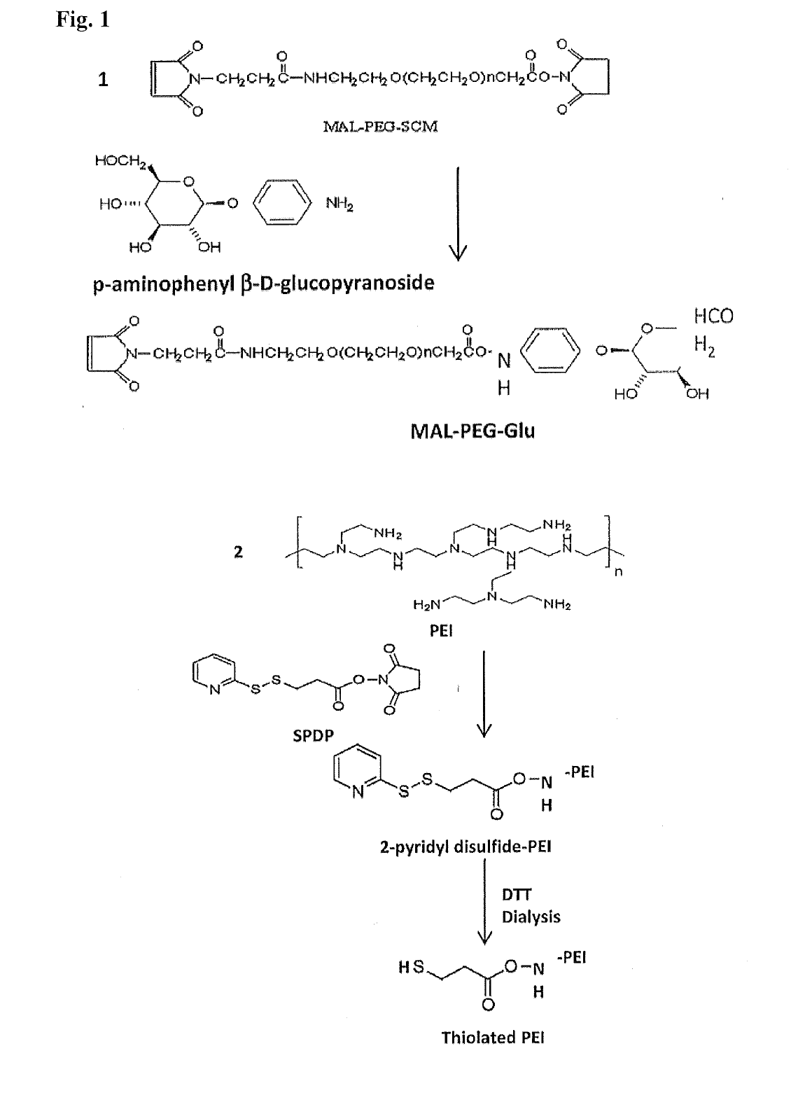

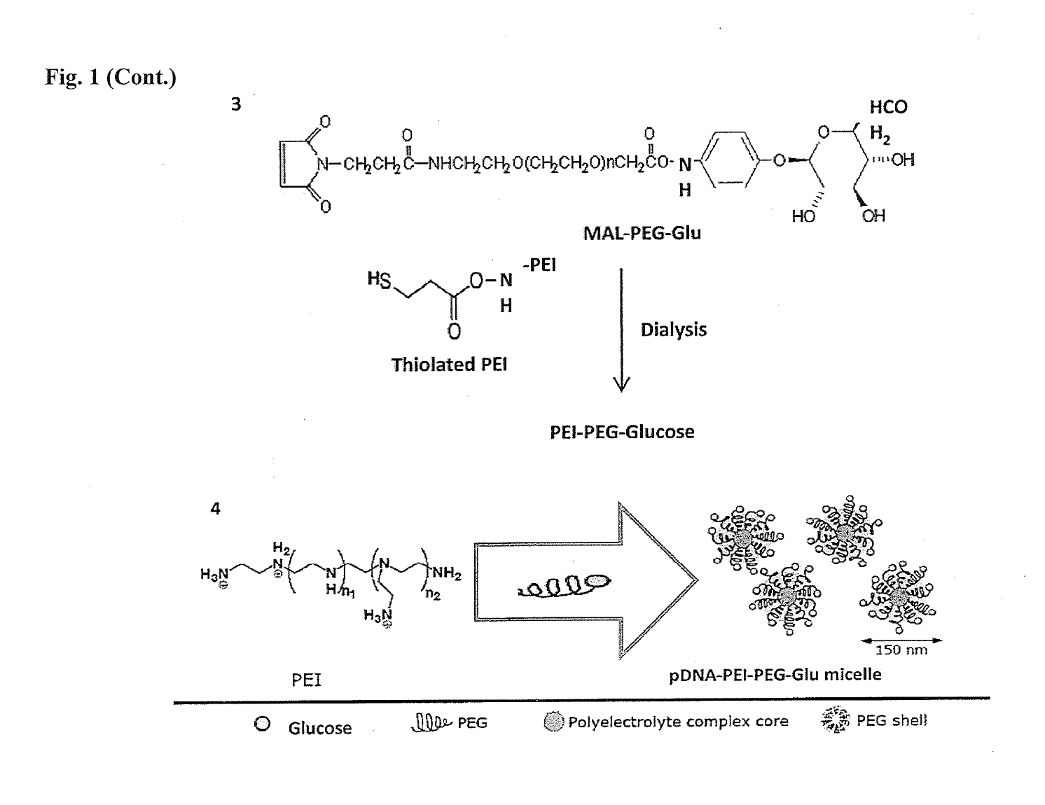

As shown in FIG. 1, a four step reaction was designed to synthesize the GLU-PEG-PEI plasmid complex. The ratio of glucose over PEG was determined by using photospectrometry at 285 nm. The optimal composition of GLU-PEG/PEI/DNA was determined to be 3:1:1.

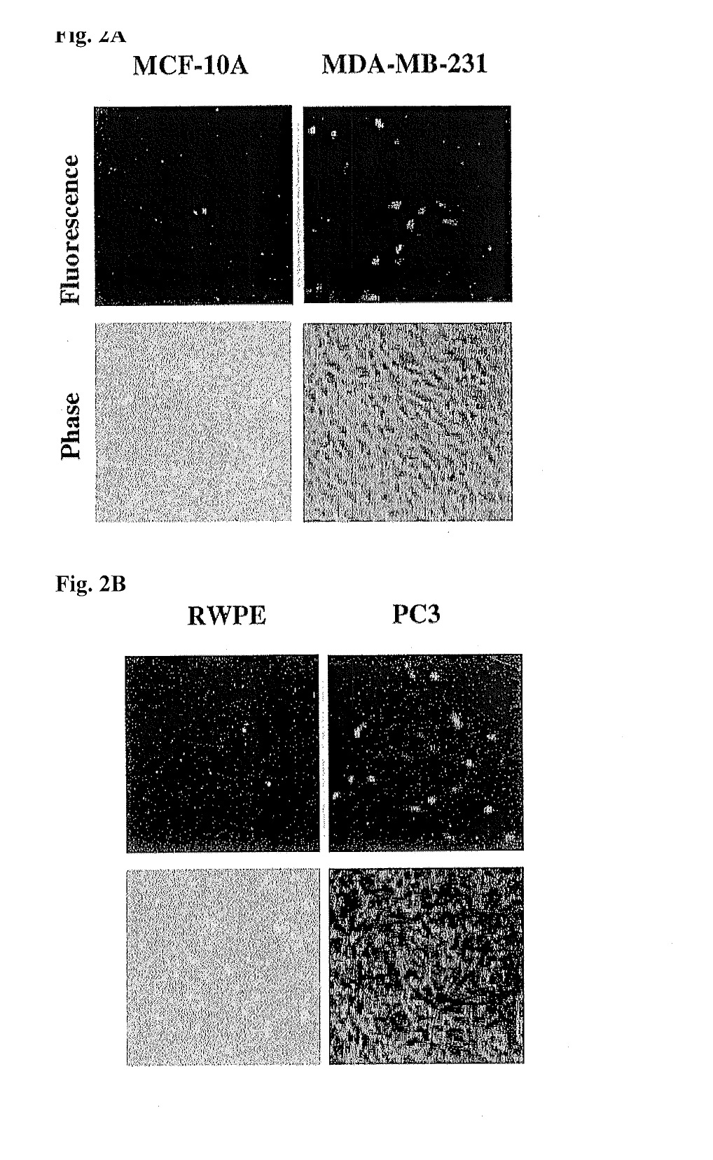

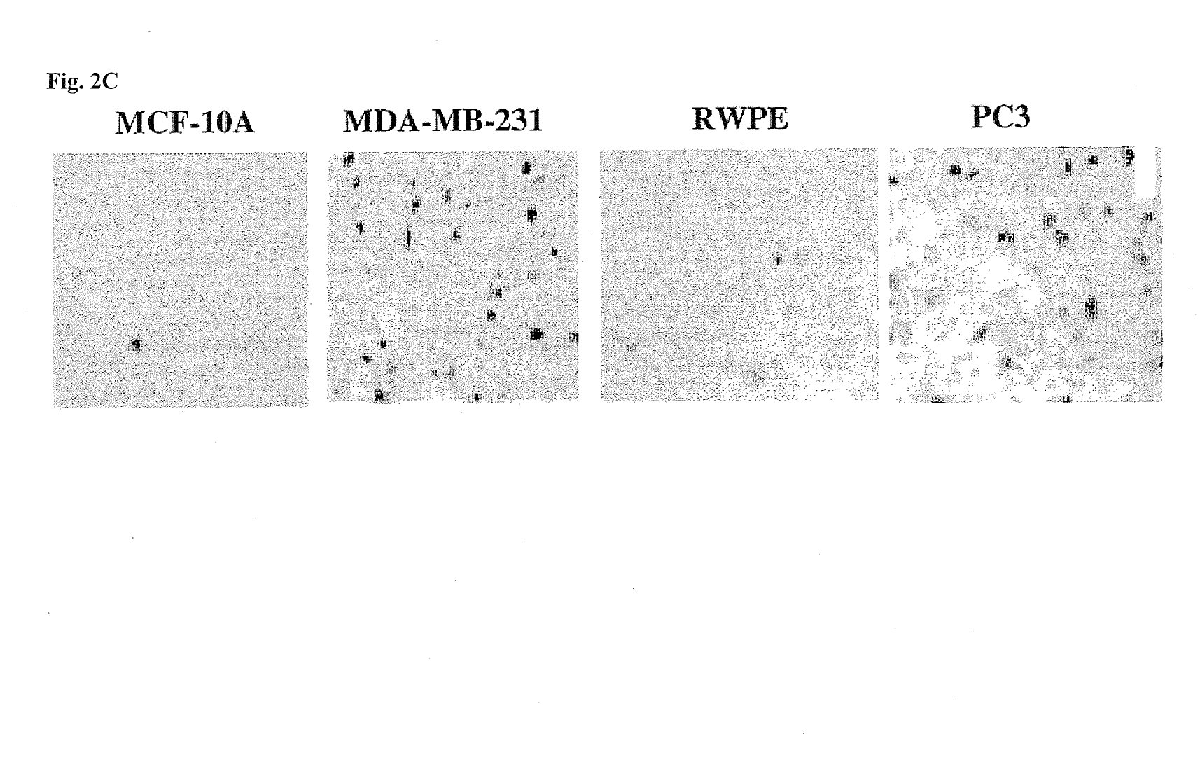

Having the GLU-PEG-PEI nanoparticles made, an EGFP expression plasmid was used to test the ability of the delivery system to preferentially target transformed over non-transformed cells for GFP expression. Specifically, the breast carcinoma cell line MDA-MB-231 was compared with the MCF-10A cell line, which is a non-transformed human breast epithelial cell line. Examination of green fluorescent signal revealed a clear differential expression of GFP in the two cell lines (FIG. 2A). On average, greater than 50% transfection efficiency was achieved in MDA-MB-231 cells whereas fewer than 5% of MCF-10A cells were found to be GFP-positive under the same conditions.

To determine whether the GLU-PEG-PEI-mediated differential delivery is cell type specific, a pair of human prostate epithelial cell lines was examined in parallel. As shown in FIG. 2B, a preferential targeting of prostate cancer cells over non-transformed prostate epithelial cells is evident. PC3, a prostate carcinoma cell line, exhibited approximately 60% GFP-positive cells. In contrast, RWPE, a non-transformed prostate epithelial cell line, had less than 5% of the cell population expressing GFP.

To further validate the results, the EGFP plasmid was replaced with a vector expressing β-galactosidase. A result similar to GFP was observed, as shown in FIG. 2C; GLU-PEG-PEI targeted cancer cells for β-gal expression. Collectively, these data demonstrated that GLU-PEG-PEI is capable of delivery of expression plasmids preferentially to carcinoma cells over non-transformed cells.

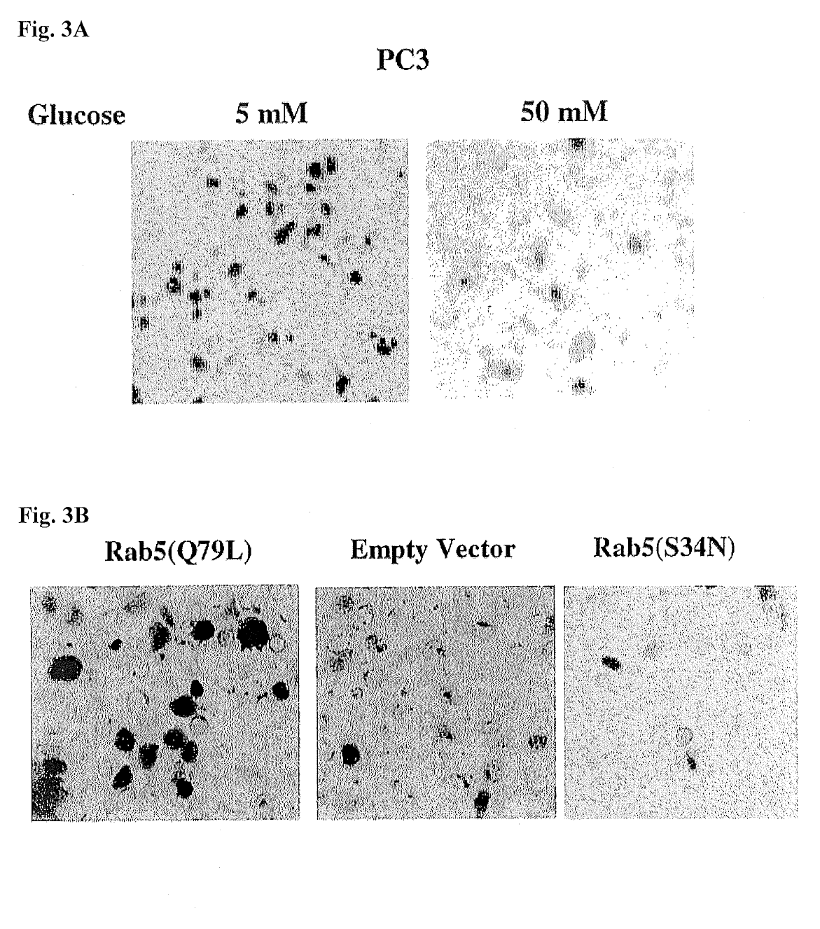

It has been well documented that cancer cells in general express high levels of glucose transporters, which are responsible for highly increased glucose uptake. To determine whether GLU-PEG-PEI utilized the glucose transporter for gene delivery, a competition experiment was performed by including an excess amount of glucose. Analysis of gene expression in the presence of a high concentration of glucose (50 mM) supports glucose transporter-dependent gene delivery, as evidenced by a marked suppression of GFP expression when compared with cells cultured with medium containing 5 mM glucose (FIG. 3A).

While the glucose competition study established that GLU-PEG-PEI-mediated gene delivery is glucose transporter-dependent, it remained unclear how the GLU-PEG-PEI/DNA complex entered cells. Given the greater size of GLU-PEG-PEI/DNA than that of glucose, it would be expected that the nanoparticle/DNA complexes enter cells via a mode different from glucose, which crosses the plasma membrane via glucose transporter-facilitated diffusion. Endocytosis-mediated internalization of receptor-ligand complex has been shown to be a primary route of cell entry for ligand-conjugated nanoparticles, including transferrin and folate. Studies were carried out to determine whether endocytosis was also the mechanism of cell entry for the GLU-PEG-PEI/DNA complex. To address this, the activity of endocytosis was altered through expression of constitutive active or dominant negative mutant Rab5, which controls the activity of early endosomes and thereby the activity of endocytosis. Consistent with an endocytosis-mediated uptake, expression of constitutive active Rab5 {Rab5(Q79L)} was associated with a marked increase in gene delivery by GLU-PEG-PEI, as demonstrated by a higher level of (3-Gal expression when compared with control (FIG. 3B). In line with these data is the observation that inhibition of endocytosis activity by expressing the dominant negative mutant {Rab5(S34N)} resulted in suppression of GLU-PEG-PEI-mediated β-Gal expression (FIG. 3B).

### Example II

### In Vivo Studies

Nanoparticles comprising GLU-PEG-PEI will be used to deliver the \tilde{□} galactosidase expression plasmid to mice for a tissue distribution study. Athymic male and female nude mice (Balb c nu/nu, 4-6 weeks old) will be purchased from Harlan laboratories. Mice will be housed under pathogen-free conditions and maintained on a 12 h light/12 h dark cycle, with food and water supplied ad libitum. Inoculums of 5×10⁶ tumor cells (e.g., lung carcinoma cells, breast cancer cells, prostate cancer cells, leukemia cells, lymphoma cells, etc.) in 0.1 ml of PBS will be mixed with Matrigel at 4° C. and then injected into the subcutaneous (s.c.) space on the right flank of mice. When tumor size reaches 1 cm, GLU-PEG-PEI/β-Gal (50 ug/DNA in 50 ul PBS) will be administered via tail vein injection or 0.5 ml of GLU-PEG-PEI/β-gal/hydrogel (20 wt % Pluronic F127 gel) mixture will be administered subcutaneously to the tumor-bearing mice. After 24 or 48 h, mice will be sacrificed by cervical decapitation. Various tissues, including liver, lung, spleen, kidney, heart, and tumor, will be collected. Frozen tissue sections will be prepared for β-Gal staining. Relative gene transfer efficiency (β-Gal expression in tumor tissues versus normal tissues) will be assessed and optimized to ensure a selective delivery of the plasmid to tumor cells.

### Example III

### Delivery of Nanoparticles to Human Subjects

Nanoparticles of this invention comprising a polynucleotide will be delivered to human subjects (e.g., orally and/or intravenously and/or subcutaneously) in a dose as described herein, depending on the composition of the nanoparticle and the particular disorder to be treated. Efficacy of treatment will be monitored by measuring changes in tumor size and/or tumor growth rate, measuring apoptosis and/or senescence of tumor cells, analyzing production of a product encoded by a nucleic acid carried by a nanoparticle, measuring cancer antigen levels (e.g., CEA, PSA), evaluating modulation of signs and symptoms associated with a subject's cancer, etc., as would be well known in the art.

### Example IV

### Dose Dependent Reduction of Tumor Size Using Nanoparticles Delivering Nucleic Acid Encoding Tumor Suppressor PTEN

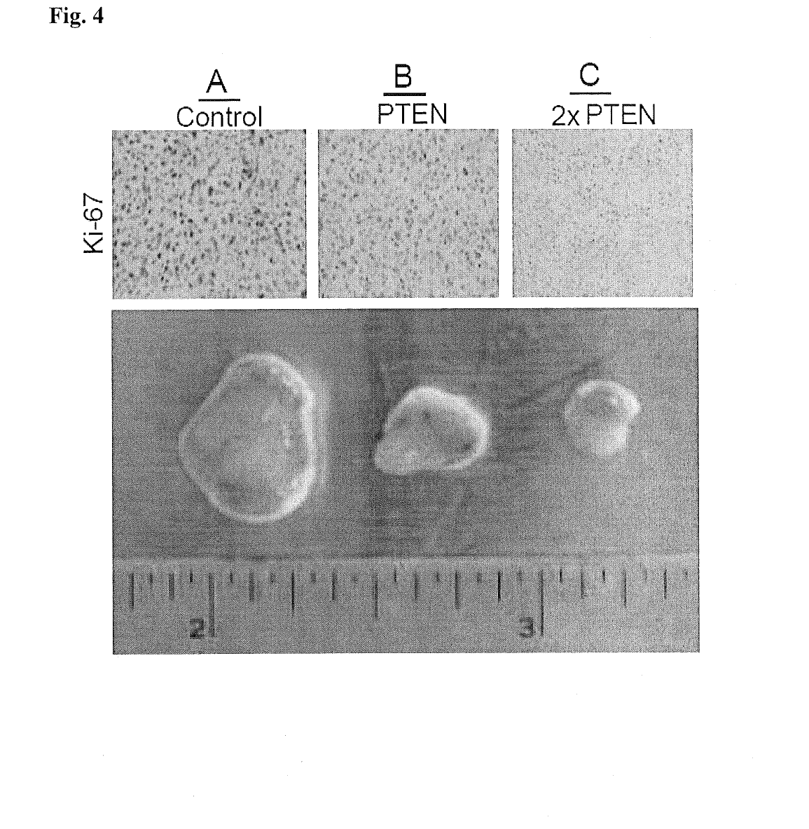

FIG. 4B shows the images of a tumor isolated from mice that were given either control vector, 20 μg of vector encoding tumor suppressor PYM or 40 μg or vector encoding tumor suppressor PTEN. Consistent with the dose dependent tumor size reduction, Ki67 staining also showed inhibition of cell proliferation by the expression of the tumor suppressor gene in a dose-dependent manner (FIG. 4A).

### Example V

### Studies on p53 Chimera Comprising p73OD

Tumor cells harbor persistent p53-activating signals because of genomic instability and oncogene activation, making restoration of p53 function an attractive approach of cancer therapy. Various methods have attempted to replace mutant p53 with its wild-type counterpart. However, due to the dominant-negative activity of the highly abundant mutant p53 proteins in tumor cells, wild-type p53 must be expressed at extremely high levels to be functional, which will likely cause severe toxicity to normal tissues. In the present invention, a p53 chimera that contains the p73 oligomerization domain (OD), is provided (p53/p73OD). Because of a lack of interaction between the p53OD and the p73OD, p53/p73OD does not associate with mutant p53 and hence is capable of effectively restoring p53 function regardless of high levels of mutant p53. Importantly, expression of p53/p73OD is associated with a marked decrease of mutant p53 protein level because of induction of MDM2 expression. As a result, a considerably lower level of expression of this chimera can adequately suppress cancer cell proliferation. In particular embodiments, the present invention also provides a glucose-conjugated PEI-nanoparticle system to preferentially deliver p53/p73OD to cancer cells. Preliminary data has provided proof-of-principle for cancer cell-specific delivery. It is thus contemplated in the present invention to utilize this novel delivery system for the development of an effective p53-based anticancer therapy with the goal of rapid translation into the clinical setting. This approach not only has the potential to revolutionize p53-based cancer therapy but also to provide a cancer cell-specific delivery system that has a broad use in cancer therapeutics.

### Preliminary Studies

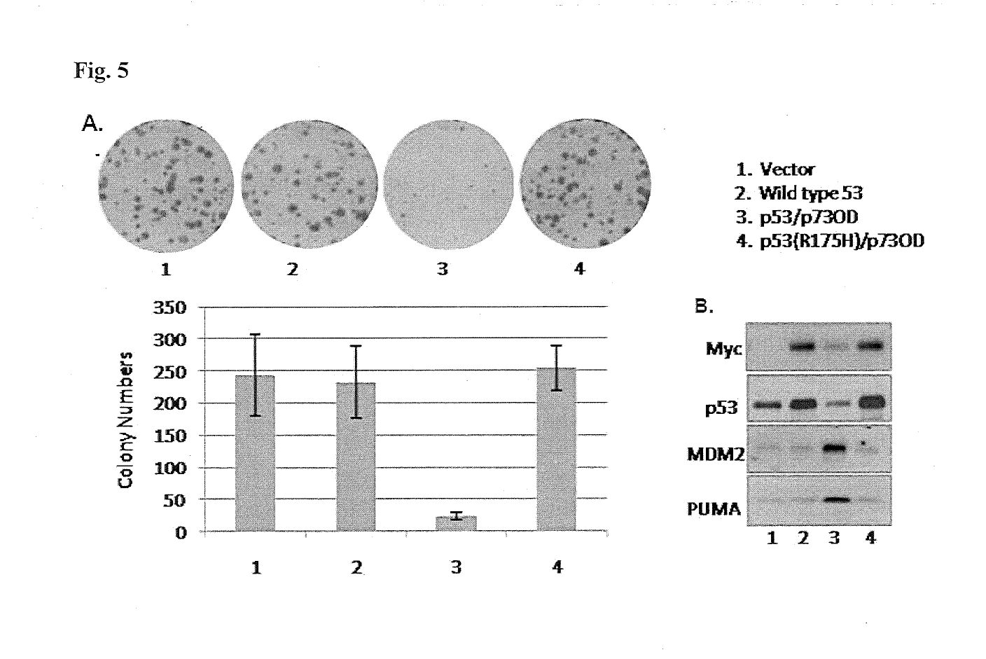

Studies with mouse tumor models showed that restoring p53 expression caused tumor regression, indicating that established tumors are vulnerable to p53 [26, 27]. However, more than half of human cancers express high levels of mutant p53 proteins, which effectively inactivate wild-type p53 via OD-mediated association, making restoration of p53 function in tumor cells very difficult. The present invention utilizes a p53/p73 chimera to circumvent this OD-mediated inactivation by mutant p53. A series of p53/p73 chimeric proteins were generated by swapping corresponding domains of p53 and p73 [28]. Studies of these chimeric proteins showed that p53/p73OD does not bind to p53; rather it associates with p′73, indicating that p53 and p73 will not associate with each other via the oligomerization domain (OD). A crystal structure study has confirmed this observation [29]. Functional characterization showed that p53/p73OD preserves all the functions of p53 tested [28]. When expressed in MDA-MB-231 cells (a breast cancer cell line that expresses a high level of mutant p53) p53/p73OD, but not wild-type p53, effectively inhibited cell growth (FIG. 5A), p53/p73OD also induced a significant decrease of mutant p53 protein level (FIG. 5B, lane 3), likely resulting from the induction of MDM2 expression. This is of particular significance considering the dominant-negative and gain-of-function activities of mutant p53 that are primarily responsible for the oncogenic function. The results demonstrate that, unlike wild-type p53, p53/p73OD can induce efficient growth suppression in the presence of a high level of mutant p53, implicating that expression of this chimera can serve as an effective approach to restore p53 function in mutant p53 expressing tumor cells.

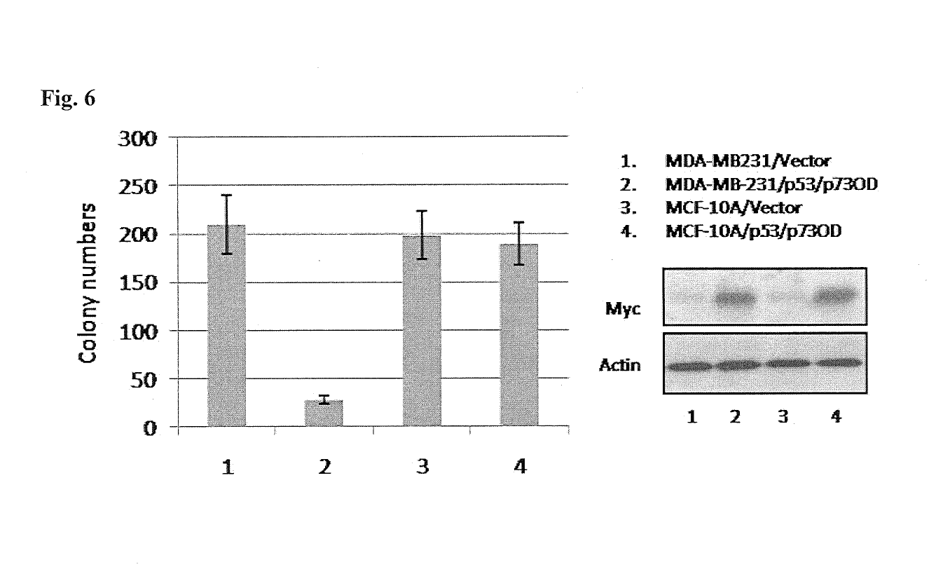

Contrary to tumor cells, normal cells do not usually harbor intrinsic signals of p53 activation. As a result, the response of normal cells to a moderate level of p53/p73OD expression is likely to differ from that of cancer cells. This was tested by comparing MDA-MB-231 cells and MCF-10A cells (a non-transformed human breast epithelial cell line), using a similar colony formation assay. The result indicates that when expressed at the level of inducing marked inhibition of MDA-MB-231 cell growth, p53/p73OD did not significantly impact the growth of MCF-10A cells (FIG. 6).

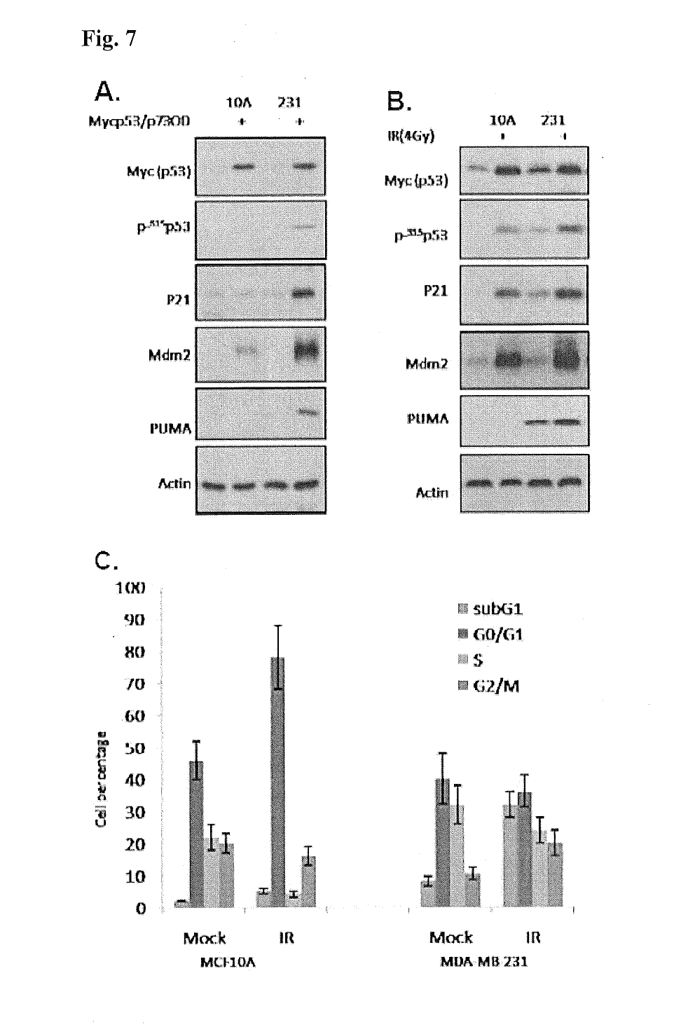

To understand potential mechanisms underlying the differential sensitivity of cancer cells and non-transformed cells to p53/p73OD expression, p53 activity was examined further. Western blot with an anti-Sp15p53 antibody, a commonly used surrogate marker of p53 activation, indicated that p53/p73OD was phosphorylated in MDA-MB-231 cells but not in MCF-10A cells (FIG. 7A). In accord with the phosphorylation, p53/p73OD expression resulted in a robust induction of PUMA, MDM2 and p21 expression in MDA-MB-231 but not MCF-10A cells (FIG. 7A), consistent with the different sensitivity shown in FIG. 6. The result is in line with the notion that cancer cells, but not normal cells, harbor strong intrinsic p53-activating signals. The low activity of p53/p73OD in MCF-10A cells led to the question of whether the chimera could be activated by exogenous stimuli such as radiation. When treated with IR, p53/p73OD phosphorylation was markedly induced in MCF-10A cells, as well as in MDA-MB-231 cells, indicative of activation of the chimera by DNA damage in both cell types (FIG. 7B). Flow cytometry analysis revealed a significant G1 cell cycle arrest in IR-treated MCF-10A cells. In contrast, MDA-MB-231 cells exhibited little G1 cell cycle arrest and rather showed a marked increase of sub-G1, or apoptotic population (FIG. 7C). This result implicates a differential response of transformed versus non-transformed cells to DNA damage-induced activation of p531p73OD.

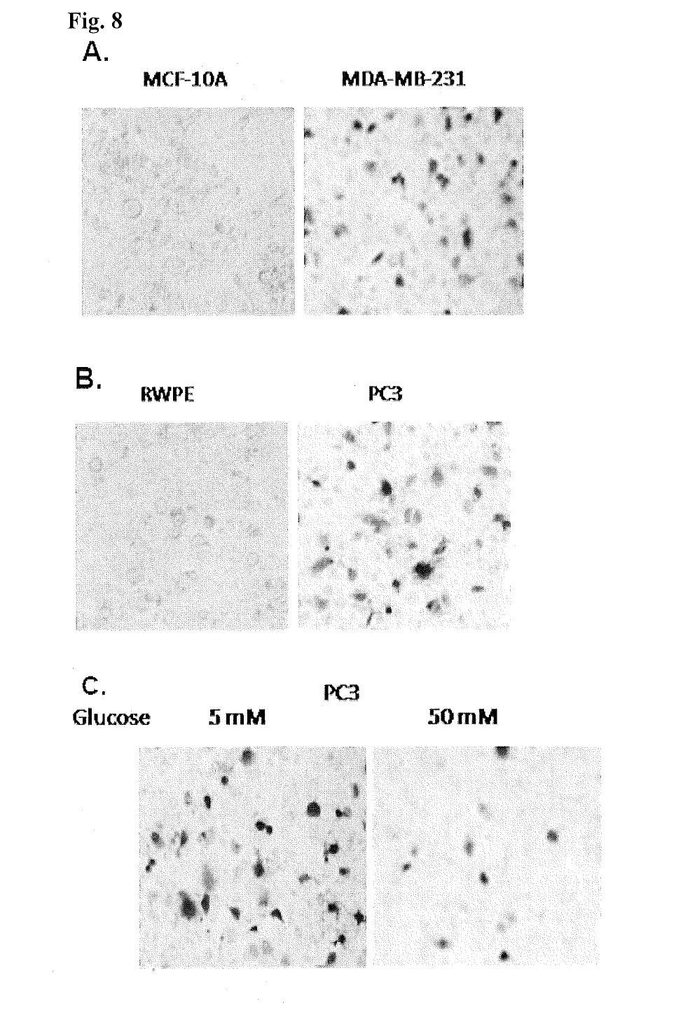

Having shown that p53/p73OD can effectively restore p53 function in cancer cells, strategies of gene delivery have been explored. Over the years, various delivery systems, such as cationic lipids, cationic polymers and viral vectors, have been introduced. Whereas viral vectors can provide a high efficiency of gene transfer, immunogenicity and potential of insertional mutagenesis in the host genome are of major concern. As a result, non-viral delivery systems have become increasingly popular. In the studies described herein, cationic polymers were used to develop a novel form of cancer cell-targeting by exploiting the Warburg effect, i.e., cancer cells take up more glucose than healthy cells. Specifically, glucose-conjugated PEG-linked polyethyleneimine (PEI) (GLU-PEG-PEI) was synthesized. As shown in FIG. 1, a 4-step reaction was designed to synthesize GLU-PEG-PEI plasmid complex. The ratio of glucose over PEG was determined by using photospectrometry at 285 nm. The optimal composition of GLU-PEG/PEI/DNA was determined to be 3:3:1. A β-galactosidase expression (a lacZ plasmid) system was used to test the ability of the delivery system to preferentially target transformed over non-transformed cells for β-gal expression. Specifically, the breast carcinoma cell line MDA-MB-231 was compared with MCF-10A cells. 5-bromo-4-chloro-3-indolyl-β-D-galactopyranoside (X-gal®) staining revealed a clear differential expression of β-gal in the two cell lines (FIG. 8A). On average, greater than 60% transfection efficiency was attained in MDA-MB-231 cells whereas fewer than 5% of MCF-10A cells were found β-gal positive under the same condition. To determine whether the GLU-PEG-PEI-mediated differential delivery is cell type specific, a pair of human prostate epithelial cell lines were examined in parallel. As shown in FIG. 8B, a preferential targeting of prostate cancer cells over non-transformed prostate epithelial cells is evident. PC3, a prostate carcinoma cell line, exhibited approximately 60% β-gal positive cells. In contrast, RWPE, a non-transformed prostate epithelial cell line, had less than 5% of the population expressing β-gal. To determine whether the GLU-PEG-PEI utilized the glucose transporter for gene delivery, a competition experiment was performed by including an excess amount of glucose. Analysis of gene expression in the presence of high concentration of glucose (50 mM) supports a glucose transporter-dependent gene delivery, as evidenced by a marked suppression of β-gal expression when compared with cells cultured with medium containing 5 mM glucose (FIG. 8C). Together, these data demonstrated that GLU-PEG-PEI is capable of delivering expression plasmids preferentially to carcinoma cells over non-transformed cells.

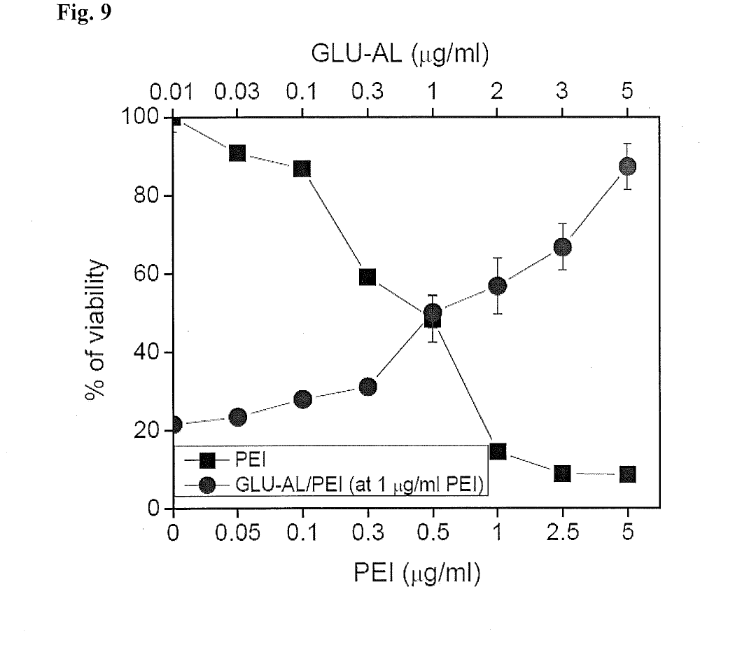

While PEI can provide excellent transfection efficiency, its usage can be hampered by the high cellular toxicity. Indeed, PEI negatively affected MDA-MB231 cell viability in a dose dependent manner (FIG. 9). However, the cytotoxicity was found to be reduced when PEI was mixed with GLU-PEG or GLU-ALG. This effect was confirmed by using increasing amounts of GLU-ALG, which resulted in a dose-dependent decrease of cytotoxicity (FIG. 9).

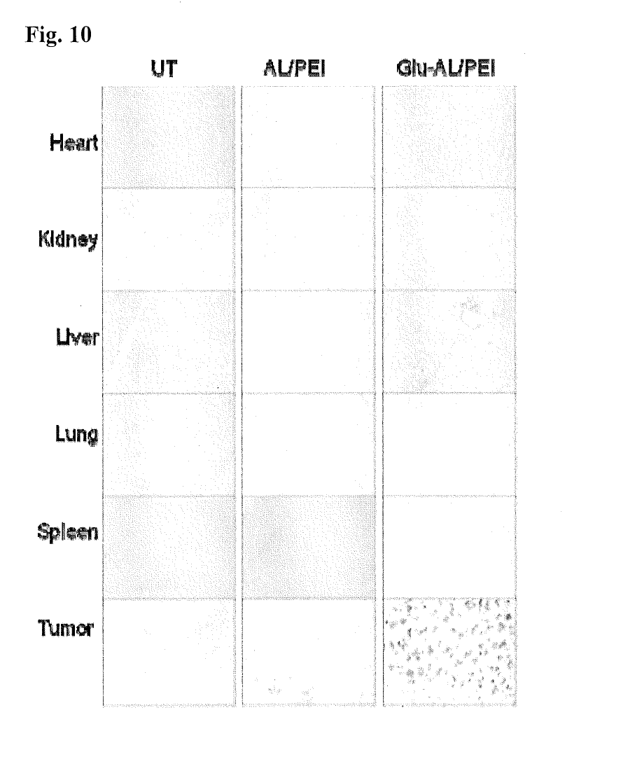

A tumor xenograft mouse model was used to assess the ability of GLU-ALG-PEI to deliver plasmids selectively to tumor cells in vivo. Inoculums of 5×10⁶ MDA-MB-231 cells in 0.1 ml of PBS were mixed with Matrigel at 4° C. and then injected into the subcutaneous (s.c.) space on the flanks of mice. When tumor size reached approximately 200 mm, GLU-PEG-PEI/lacZ in PBS was administered via tail vein injection to the tumor-bearing mice. After 48 h, mice were sacrificed by cervical decapitation. Various tissues, including liver, lung, spleen, kidney, heart, and tumor were collected. Frozen tissue sections were prepared for β-Gal staining. Strikingly, β-Gal positive staining was observed only in the tumor tissue but not other tissues (FIG. 10). The failure of ALG-PEI to deliver lacZ under the same condition indicates a glucose-dependent tumor cell targeting.

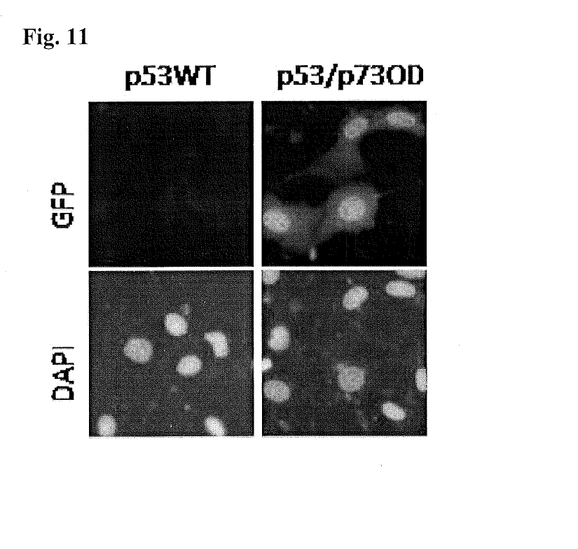

To facilitate the detection of p53 activation in situ, a Pg13-GFP system was developed in which 13 repeats of a canonical p53 responsive element were cloned to drive the expression of GFP protein. An absolute dependency of the GFP expression on p53 was verified with p53-null H1299 cells. MDA-MB-231 cells stably expressing the Pg13-GFP construct were generated and incubated with GLU-PEG-PEI/WTp53 or p53/p73OD. The cells were fixed 24 h later. DAPI staining was performed to facilitate the detection of cell nuclei and the GFP signals were examined under a fluorescence microscope. p53/p73OD, but not p53 wt, was associated with an increase of p53 transcription activity in MDA-MB-231 cells as evidenced by GFP expression (FIG. 11). The result indicates that the GLU-PEG-PEI particles successfully delivered p53/p73OD, which was not only expressed but also functional. Pg13-lacZ and Pg13-Luciferase plasmids have been created and will be used in parallel with the Pg13-GFP for the in vivo study of GLU-PEG-PEI/p53/p73OD.

### Experimental Models.

The studies described herein will use both in vitro and in vivo models. The cellular models include NCI60 human cancer cell lines and other non-transformed or cancer cell lines obtained, e.g., through the American Type Culture Collection (ATCC). Nude mice will be used to create tumor xenograft models. Genetically engineered mouse models of human cancer will also be used.

Studies to Test p53/p73OD in NCI60 Human Tumor Cell Lines for its Activity to Inhibit Cell Proliferation

To validate the growth inhibitory activity of p53/p73OD on a broader basis, studies will be carried out on NCI60 human cancer cell lines that have been derived from common human tumors such as lung, colon, breast and prostate. The activity of p53/p73OD in cancer cell lines will be assessed 1) at the biochemical level-the expression of the chimeric protein and the p53 target genes and 2) at the cellular level-cancer cell proliferation via the AlamarBlue® and colony survival assays. It has been well established that p53 inhibits cell proliferation by induction of either cellular senescence or apoptosis, dependent on cell types. These two cellular responses will be examined by using corresponding markers to determine the mechanism underlying p53/p73OD-mediated growth inhibition. Retrovirus vectors (pBABE) encoding myc-tagged wild-type p53, p53/p73OD or p53(R175H)/p73OD have been prepared and will be used. The Myc-tag allows the re-introduced protein to be readily distinguished from endogenous mutant p53 protein. Wild-type p53 will be tested in parallel to assess the dominant negative activity of mutant p53 in cancer cells. Recent studies have suggested a transcription-independent role for p53 in induction of apoptosis [1, 2]. Such a possibility will be examined by including the transcription-deficient mutant p53(R175H)/p73OD.

Retroviral-mediated gene transfer will be performed as described previously [30]. Different expression levels of p53 or p53/p73OD will be obtained by infecting 25 million recipient cells with varying amount virus stock (0.5, 1, 1.5, 2, 2.5, or 3 ml) per 100 mm dish in the presence of 5 μg/ml polybrene and incubated at 37° C. Control infections with pBABE-lacZ virus performed in preliminary studies with a number of carcinoma cell lines indicate that an average of 50-80% of cells has been routinely infected. Twenty-four hours post-infection, the cells will be selected in medium containing 2-5 μg/ml puromycin (Sigma), dependent on cell types, for 2 days to eliminate uninfected cells. Cells will be recovered in puromycin-free medium for 24 h and then subject to further analysis.

An AlamarBlue assay will be performed to determine cell viability using AlamarBlue® (Invitrogen) according to manufacturer's protocol. A time course experiment (0, 1, 3, 5, 7, and 9 days) will be carried out to monitor cell viability with time.

For colony formation assays, cells will be seeded in p60-mm culture dishes (cell numbers will vary dependent on the plating efficiency of each cell type). After 12 days, colonies will be fixed and stained with crystal violet. Only colonies containing at least 50 cells will be counted, and plating efficiency (colonies counted/cells seeded) will be calculated.

Cellular senescence will be determined using a number of cellular markers including senescence-associated [symbol] galactosidase (SA-β-Gal), p15-Ink4b, p16-Ink4a, DcR2 and Dec-1. A time course similar to a cell viability experiment will be performed to examine development of a senescent phenotype. Apoptosis will be measured by flow cytometry analysis of annexin-5 positive cells and Western blot will be used for activated caspase-3 at various times as described above.

Cells will be harvested for Western analysis of protein expression using anti-Myc, p53, p21, MDM2 or actin. RNA will be isolated for gene array analysis. Specifically, the p53 pathway array (ABBioScience) will be used, which is designed to profile gene expression of a panel of 113 key genes involved in the p53 pathways. The p53 target genes regulated are divided into the functional clusters that are involved in apoptosis; the cell cycle; cell growth, proliferation and differentiation; and DNA repair. The array data will be confirmed by QRT-PCR and Western analysis.

Tumor cells are usually under significantly higher apoptotic stress than normal cells because high proliferation rate is often associated with the induction of pro-apoptotic genes [31]. Together with the fact that tumor cells harbor persistent p53-activating signals because of genomic instability and oncogene activation [32], it can be expected that p53/p73OD will be readily activated upon expression in cancer cells, as shown in the preliminary study. p53/p73OD expression is expected to result in significant suppression of proliferation in most tumor cell lines regardless of the p53 status, whereas wild type p53 will be active only in cancer cell lines that are deficient in p53 expression. Considering that mutant p53 proteins usually accumulate in tumor cells to a very high level, the ability of p53/p73OD to escape the inhibition by mutant p53 may prove to be very advantageous because a moderate level of expression is expected to be adequate for inhibition of cancer cell proliferation, thereby avoiding potentially unwanted side effects. The proposed experiments of expressing different levels of p53/p73OD should allow for a determination of the optimum level of expression adequate for suppression of tumor cell growth. A recent genetic study has shown that MDM2 is primarily responsible for the degradation of mutant p53 in cancer cells [3], which is corroborated in the preliminary study. A considerable reduction of mutant p53 protein levels in tumor cells upon expression of p53/p73OD is anticipated because of induction of MDM2 expression. This is of great significance because the high abundance of mutant p53 protein in tumor cells has been shown to be responsible for resistance to treatment and poor patient survival. Thus, introduction of p53/p73OD into tumor cells not only restores the tumor suppressor function but also eliminates the highly oncogenic mutant p53 protein. This one-stone-two-birds effect of p53/p73OD would provide a strong rationale for using the chimera as an anticancer therapy.

It has been well documented that the tumor suppressor function of p53 is primarily mediated by its transcriptional activity. By performing the p53 pathway specific gene array analysis, induction of the p53 target genes expression in cancer cells by p53/p73OD is expected to be detected. Dependent on cancer types, the magnitude and the pattern of gene expression may vary because of differences in spectrum of p53 responsive genes and availability of p53 co-activators in different tumor cells. The gene expression data and its correlation with p53/p73OD-induced growth inhibition will be analyzed in each cancer type. Such information should be informative in assessing the response of a given tumor type to p53/p73OD expression.

Studies using tumor mouse models have demonstrated that p53 reactivation caused tumor regression by induction of senescence or apoptosis, dependent on the tumor types, with mainly apoptosis in hematopoietic malignance and senescence in other tumor types [26]. The studies outlined using markers of senescence or apoptosis are expected to uncover the mechanism underlying p53/p73OD-mediated growth inhibition in each tumor cell line examined. The time course experiments should allow for a determination of the kinetics of induction of each phenotype induced by p53/p73OD.

In summary, these proposed studies will assess the ability of p53/p73OD to restore p53 function in a large panel of human cancer cell lines. p53/p73OD induced gene expression and mechanisms underlying the growth inhibition will be examined. Results obtained from the outlined studies are expected to provide strong evidence to demonstrate the superior activity of p53/p73OD to inhibit growth of tumor cells, mutant p53 expressing cells in particular.

Studies to Examine the Combined Effect of p53/p73OD Expression with Radio- or Chemotherapy

Radiation and chemotherapeutic drugs kill cancer cells primarily via induction of DNA damage, which very potently activates p53. Results from the preliminary study indicate that while p53/p73OD was activated by radiation in both MDA-MB-231 cells and MCF-10A cells, the subsequent cellular effects were quite different. In contrast to MDA-MB-231 cells where p53/p73OD activation resulted in mainly cell death, radiation induced activation of p53/p73OD in MCF-10A cells was primarily associated with G1 cell cycle arrest. Such distinct responses between transformed and non-transformed cells may provide an opportunity for differentially targeting cancer cells. This will be tested by examining the combined use of p53/p73OD with radiation and/or chemotherapeutic drugs. A low to moderate dose of radiation or chemotherapeutic drug pretreatment would be expected to induce cell cycle arrest in normal cells whereas cancer cells will continue proliferating because of defects in the cell cycle checkpoints. As a consequence, normal cells would be protected since growth arrested cells are usually more resistant to damages induced by subsequent treatments. Cancer cells, on the other hand, would be sensitized because of synergistic activation of p53/p73OD by the intrinsic oncogenic signals and exogenous DNA damage signals. This will be tested using both cell-based and animal models.

Different levels of expression of either p53/p73OD or p53 will be achieved via retroviral infection as described above in a number of paired transformed and non-transformed cells, including MDA-MB-231 and MCF-10A breast epithelial cells, PC3 and normal prostate epithelial cells, NCI-11358 and 3B3 lung epithelial cells, Ovcar-3 and IOSE-29 ovarian epithelial cells, and HT-29 and RIE-1 colon epithelial cells, which represent the major human cancer types. A dose course experiment of irradiation or chemotherapeutic drugs will be performed. Cellular responses including cell cycle arrest, cell survival and senescence will be compared in the paired cell lines. Western analysis will be carried out in parallel to monitor the p53 response. A level of p53/p73OD expression that does not cause significant cell death in carcinoma cells will be selected for pretreatment, and effects of the pretreatment on sensitivity of the paired cells to subsequent treatment of radiation or chemotherapeutic drugs will be examined.

The carcinoma cells expressing either p53/p73OD or p53 will be implanted to nude mice to generate xenograft models for study of combination with radiation or chemotherapeutic drugs in vivo. A strategy similar to that described herein in cell studies will be used.

DNA damages caused by irradiation or chemotherapeutic drugs are the most potent signal of p53 activation. Together with the persistent p53-activating signals intrinsic to cancer cells due to oncogene activation and other stress phenotypes [33], an enhanced sensitivity of cancer cells to the combination of p53/p73OD with irradiation or chemotherapeutics is anticipated. Oncogenic stress and DNA damage activate p53 via overlapping but distinct mechanisms, which may lead to synergistic activation and thereby augment the activity of p53/p73OD in cancer cells. Such an effect would enable clinicians to reduce doses of irradiation or chemotherapeutic drugs, minimizing potential side effects. The proposed studies will allow for the testing of the beneficial effect of combined use of p53/p73OD with irradiation, or chemotherapeutics. As shown in preliminary studies, radiation or chemotherapeutic drug induced activation of p53/p73OD in normal cells is associated with G1 cell cycle arrest; pretreatment with a moderate dose is expected to provide a protective effect to normal cells because growth arrested cells are usually more resistant to cytotoxic therapies. By comparing non-transformed cells among themselves and with cancer cells in the presence or absence of p53/p73OD expression, a differential sensitivity of normal and cancer cells to the treatment of anticancer therapies is anticipated. The proposed mouse xenograft models are expected to complement cell-based studies to demonstrate a superior antitumor activity of p53/p73OD, especially in mutant p53 expressing tumors. Of worthy note is localized delivery of irradiation that can limit p53 activation to the site of tumor, further minimizing the damage to normal tissue.

In summary; these studies will investigate the combined effect of chemotherapeutic drugs or radiation with p53/p73OD on cell survival. A strategy of pretreatment with a low dose that induces cell cycle arrest in non-transformed cells will be explored to enhance the therapeutic efficacy of the combined use of p53/p73OD with chemotherapy or radiation.

Studies of GLU-PEG-PEI Targeting Tumor Cells for Expression of p53/p73OD in Mouse Tumor Models.

The effectiveness of anticancer therapeutics is determined by the ability to reduce and eliminate cancer cells without damaging healthy tissues Thus, a strategy of preferentially targeting cancer cells is essential in the success of cancer therapeutics. This invention provides a novel form of cancer cell-targeting nanoparticle-based delivery system by exploiting the Warburg effect, i.e., cancer cells take up more glucose than healthy cells. Preliminary studies have shown that GLU-PEG-PEI enabled lacZ expression plasmid delivery with a considerablly greater efficiency in PC3 or MDA-MB-231 than in RWPE or MCF-10A cells, respectively. Mouse tumor models will be used to test the utility of GLU-PEG-PEI in vivo.

GLU-PEG-PEI will be used to deliver the lacZ expression plasmid for tissue distribution study. All animal experiments will follow the guidelines of the Institutional Animal Care and Use Committee of UTHSCSA. Athymic male and female nude mice (Balb c nu/nu, 4-6 weeks old) will be purchased from Harlan laboratories. Mice will be housed under pathogen-free conditions and maintained on a 12 h light/12 h dark cycle, with food and water supplied ad libitum. GLU-PEG-PEI/DNA will be suspended in sterile saline solution with a DNA concentration not beyond 200 μg/ml to avoid precipitation. A maximum tolerable dose will be determined by intravenous injection via tail vein of an amount equivalent to 20, 40, 60, 80, or 100 μg dose of plasmid DNA and mice will be closely monitored for any signs of toxicity. Animals will be weighed every 3 days. Aspartate aminotransferase (AST) and alanine aminotransferase (ALT) serum transaminase activity levels, commonly used surrogate markers of acute toxicity, will be determined from blood samples collected at 1, 3, 6, 9, 15, or 30 days after the systematic administration of the GLU-PEG-PEI/DNA nanoparticles. Liver, lung, spleen and kidney will be harvested for histological examination.

Mouse xenograft tumor models will be generated for assessing a preferential uptake of the GLU-PEG-PEI/DNA nanoparticles by tumors. Inoculums of 3×10⁶ tumor cells in 0.1 ml of PBS will be mixed with Matrigel at 4° C. and then injected into the subcutaneous (s.c.) space on the flanks of mice. When tumor size reaches 0.2 cm, GLU-PEG-PEI/lacZ in PBS will be administered via tail vein injection to the tumor-bearing mice. After 24 or 48 h, mice will be sacrificed by cervical decapitation. Various tissues, including liver, lung, spleen, kidney, heart, and tumor will be collected. Frozen tissue sections will be prepared for β-Gal staining. Relative gene transfer efficiency will be assessed and optimized to ensure a selective delivery of the plasmid to tumor cells. In parallel, the nanoparticles will be used to deliver a plasmid encoding either EGFP or firefly luciferase and visualize the distribution using low energy laser scanning for fluorescence imaging or an intraperitoneal injection of 150 mg/kg luciferin, the substrate for firefly luciferase for luminescence imaging. In addition, the GLU-PEG-PEI/DNA will be administered by intratumoral injection and the distribution of nanoparticles will be determined. The duration of gene expression will be monitored, which will serve as an important reference for determining whether multiple injection of the nanoparticles is required.

Upon the establishment of the condition of delivery, the activity of p53/p73OD will be assessed with wild type p53 or p53(R175H)/p73OD included as controls. Initial studies will be of implants to four types of carcinoma cells; MDA-MB-231, PC3, HT-29, and Ovcar-3 as the representatives of breast, prostate, colorectal and ovarian cancers, respectively. These cancer cell lines have been stably transfected with Pg13-lacZ, Pg13-GFP or Pg13-Luc and they will be used for xenograft model generation. When tumor size reaches 1 cm, GLU-PEG-PEI/DNA complex in PBS will be administered via tail vein injection. The tumor bearing mice will be monitored for an additional 4-8 weeks. Body weight and tumor volumes will be measured every third day. Tumor volume will be calculated using the equation: (volume=length×width×depth×0.5236 mm³). Fluorescence or luminescence imaging will be used to monitor the kinetics of p53/p73OD-mediated tumor suppression in the xenograft tumors derived from Pg13-GFP or Pg13-Luc expressing cancer cells. Based on the correlation between p53/p73OD expression and the extent of tumor regression, a determination will be made of whether additional administration of GLU-PEG-PEI/p53/p73OD is necessary. At the end of each experiment, mice will be sacrificed by cervical decapitation. Tumor samples and selective tissues will be collected for analysis of gene expression as described herein and histological examination using markers of apoptosis and senescence will be carried out as also described herein.

In parallel, the mouse xenograft models will be treated with either chemotherapeutic drugs or radiation to assess the combined effect of GLU-PEG-PEI/p53/p73OD with chemotherapy or radiation on tumor growth.

The results derived from xenograft mouse models will be validated by testing p53/p73OD with genetically engineered mouse models of human cancer from NIH/NCI Mouse Models of Human Cancer Consortium (http://mouse.ncifcrf.gov).

GLU-PEG-PEI is expected to preferentially target tumor cells for delivery in xenograft mouse models. The PEG coating can render PEI significantly increased in vivo circulation time. In addition, PEG has been used as a pharmaceutical excipient and is known to be non-toxic and non-immunogenic [34]. GLU-PEG-PEI/DNA nanoparticles are expected to show little toxicity and be well tolerated by mice. Preliminary data from cell-based studies have demonstrated proof-of-principle for the GLU-PEG-PEI/DNA nanoparticles that target cancer cells in a glucose transporter-dependent fashion. These studies of xenograft mouse models are expected to demonstrate a preferential uptake of the nanoparticles by tumors. The use of plasmids encoding EGFP or firefly luciferase should allow for monitoring GLU-PEG-PEI-mediated cancer cell delivery. Real-time luminescence and fluorescence (400-900 nm) imaging will be used to monitor and record the distribution of the nanoparticles within a living mouse. When complemented with the proposed histological examination of β-gal signal, these in vivo image methods should allow for a determination of temporal and spatial distributions of the expression plasmid in tumor cells. A minimum level of distribution of the nanoparticles to normal tissues is anticipated, the expression of EGFP or luciferase should allow for the detection of which tissues the plasmid may express and for the quantification of the level of expression. Such information would be instrumental in guiding the use of p53/p73OD. The use of Pg13-EGFP or Pg13-Luc stably expressing carcinoma cells will prove to be advantageous in monitoring the in vivo activity of p53/p73OD, which when correlated with tumor regression, should allow a determination of the optimal p53/p73OD dose and duration of expression for the best therapeutic outcome. When combined with chemo or radiation therapy, GLU-PEG-PEI-mediated cancer cell delivery of p53/p73OD is expected to selectively sensitize tumor cells to the therapeutic effect of chemotherapy or radiation, which should allow reducing doses of the treatments, further minimizing potential side effects.

A dual functional nanoparticles for simultaneous imaging and therapeutic applications can also be developed. In a preliminary study, alginate-fluorescein isothiocyanate (FITC)/Glu-PEG-PEI nanoparticles were synthesized and are currently under study for these applications.

In summary, studies will be carried out to explore experimental therapeutics by focusing on two areas, which are related but not mutually dependent: 1) To develop a novel p53-based cancer therapy that is highlighted by the use of the p53/p73OD chimera that is resistant to inactivation by mutant p53. Preliminary data have demonstrated superior activity of this chimera in restoration of p53 function in mutant p53 expressing cancer cells. Considering that more than half of human cancers harbor high levels of mutant p53, the use of p53/p73OD is expected to have significant therapeutic potential, either alone or in combination with chemotherapy or radiation. 2) To develop a nanoparticle-based cancer cell-targeting delivery system. Data from preliminary studies have provided proof-of-principle for the GLU-PEG-PEI nanoparticles as a cancer cell-selective delivery method. While designed for enhancing the therapeutic efficacy of p53/p73OD in some embodiments, the delivery system is expected to have a wide application in additional embodiments for improving cancer therapeutics.

All publications, patent applications, patents, references for nucleotide sequences, references for amino acid sequences and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

### REFERENCES

1. Prives, C. and P. A. Hall, The p53 pathway. J Pathol, 1999. 187(1): p. 112-26.
2. Vousden, K. H., p5.3; death star. Cell, 2000. 103(5): p. 691-4.
3. Terzian, T., et al., The inherent instability of mutant p53 is alleviated by Mdm2 or p16INK4a loss. Genes Dev, 2008. 22(10): p. 1337-44.
4. Peller, S, and V. Rotter, TP53 in hematological cancer: low incidence of mutations with significant clinical relevance. Hum Mutat, 2003. 21(3): p. 277-84.
5. Schuijer, M. and E. M. Berns, TP53 and ovarian cancer. Hum Mutat, 2003. 21(3): p. 285-91.
6. Iacopetta, B., TP53 mutation in colorectal cancer. Hum Mutat, 2003. 21(3): p. 271-6.
7. Blons, H. and P. Laurent-Puig, TP53 and head and neck neoplasms. Hum Mutat, 2003. 21(3): p. 252-7.
8. Shelling, A. N., Role of p53 in drug resistance in ovarian cancer. Lancet, 1997. 349(9054): p. 744-5.
9. Hamada, M., et al., The p53 gene is a potent determinant of chemosensitivity and radiosensitivity in gastric and colorectal cancers. J Cancer Res Clin Oncol, 1996. 122(6): p. 360-5.
10. Preudhomme, C. and P. Fenaux, The clinical significance of mutations of the P53 tumour suppressor gene in haematological malignancies. Br J Haematol, 1997. 98(3): p. 502-11.
11. Soussi, T. and C. Beroud, Assessing TP53 status in human tumours to evaluate clinical outcome. Nat Rev Cancer, 2001. 1(3): p. 233-40.
12. Legros, Y., et al., Mutations in p53 produce a common conformational effect that can be detected with a panel of monoclonal antibodies directed toward the central part of the p53 protein. Oncogene, 1994. 9(12): p. 3689-94.
13. Ory, K., et al., Analysis of the most representative tumour-derived p53 mutants reveals that changes in protein conformation are not correlated with loss of transactivation or inhibition of cell proliferation. EMBO J, 1994. 13(15): p. 3496-504.
14. Hinds, P. W., et al., Mutant p53 DNA clones from human colon carcinomas cooperate with ras in transforming primary rat cells: a comparison of the “hot spot” mutant phenotypes. Cell Growth Differ, 1990. 1(12): p. 571-80.
15. Soussi, T. and C. Beroud, Significance of TP53 mutations in human cancer; a critical analysis of mutations at CpG dinucleotides. Hum Mutat, 2003. 21(3): p. 192-200.
16. Lane, D. P. and S. Benchimol, p53: oncogene or anti-oncogene? Genes Dev, 1990. 4(1): p. 1-8.
17. Dittmer, D., et al., Gain of function mutations in p53. Nat Genet, 1993. 4(1): p. 42-6.
18. Gaiddon, J., et al., Use of biometry and keratometry for determining optimal power for intraocular lens implants in dogs. Am J Vet Res, 1991. 52(5): p. 781-3.
19. Di Como, C. J., C. Gaiddon, and C. Prives, p73 function is inhibited by tumor-derived p53 mutants in mammalian cells. Mol Cell Biol, 1999. 19(2): p. 1438-49.
20. Marin, M. C., et al., A common polymorphism acts as an intragenic modifier of mutant p53 behaviour. Nat Genet, 2000. 25(1): p. 47-54.
21. Strano, S., et al., Physical and functional interaction between p53 mutants and different isoforms of p73. J Biol Chem, 2000. 275(38): p. 29503-12.
22. Song, H., M. Hollstein, and Y. Xu, p.53 gain-of-function cancer mutants induce genetic instability by inactivating ATM. Nat Cell Biol, 2007. 9(5): p. 573-80.
23. Olivier, M., et al., TP53 mutation spectra and load: a tool for generating hypotheses on the etiology of cancer. IARC Sci Publ, 2004(157): p. 247-70.
24. Farokhzad, O. C. and R. Langer, Impact of nanotechnology on drug delivery. ACS Nano, 2009. 3(1): p. 16-20.
25. Byrne, J. D., T. Betancourt, and L. Brannon-Peppas, Active targeting schemes for nanoparticle systems in cancer therapeutics. Adv Drug Deliv Rev, 2008. 60(15): p. 1615-26.
26. Xue, W., et al., Senescence and tumour clearance is triggered by p53 restoration in murine liver carcinomas. Nature, 2007. 445(7128): p. 656-60.
27. Ventura, A., et al., Restoration of p53 function leads to tumour regression in vivo. Nature, 2007. 445(7128): p. 661-5.
28. Gu, J., et al., Identification of a sequence element from p53 that signals for Mdm2-targeted degradation. Mol Cell Biol, 2000. 20(4): p. 1243-53.
29. Joerger, A. C., et al., Structural evolution of p53, p63, and p73: implication for heterotetramer formation. Proc Natl Acad Sci USA, 2009. 106(42): p. 17705-10.
30. Tsai, K. K., et al., Cellular mechanisms for low-dose ionizing radiation-induced perturbation of the breast tissue microenvironment. Cancer Res, 2005. 65(15): p. 6734-44.
31. Nahle, Z., et al., Direct coupling of the cell cycle and cell death machinery by E2F. Nat Cell Biol, 2002. 4(11): p. 859-64.
32. Martins, C. P., L. Brown-Swigart, and G. I. Evan, Modeling the therapeutic efficacy of p53 restoration in tumors. Cell, 2006. 127(7): p. 1323-34.
33. Luo, J., N. L. Solimini, and S. J. Elledge, Principles of cancer therapy: oncogene and non-oncogene addiction. Cell, 2009. 136(5): p. 823-37.
34. Duncan, R., Polymer conjugates as anticancer nanomedicines. Nat Rev Cancer, 2006. 6(9): p. 688-701.

1. A nanoparticle selected from the group consisting of:
A) a nanoparticle comprising:
a) a polycation/polyalkylene glycol/glucose conjugate; and
b) an active agent;
B) a nanoparticle comprising:
a) a core comprising an active agent; and
b) a glucose/polyalkylene glycol conjugate surrounding the core of (a);
C) a nanoparticle comprising:
a) a core comprising a polycation and an active agent; and
b) a glucose/polyalkylene glycol conjugate surrounding the core of (a);
D) a nanoparticle comprising:
a) a polycation/alginate/glucose conjugate; and
b) an active agent;
E) a nanoparticle comprising:
a) a core comprising an active agent; and
b) a glucose/alginate conjugate surrounding the core of (a);
F) a nanoparticle comprising:
a) a core comprising a polycation and an active agent; and
b) a glucose/alginate conjugate surrounding the core of (a); and
G) any combination of (A)-(F) above.

2. The nanoparticle of claim 1, wherein the polyalkylene glycol is polyethylene glycol (PEG)

3. The nanoparticle of claim 1, wherein the polycation is polyethyleneimine (PEI).

4. The nanoparticle of claim 1, wherein the active agent is selected from the group consisting of a polynucleotide, an oligonucleotide, interfering RNA, a protein, a peptide, a chemotherapeutic drug, a cytotoxic agent, a radionuclide, a detectable marker, an imaging agent and any combination thereof.

5. The nanoparticle of claim 4, wherein the polynucleotide is a plasmid encoding a p53 chimera comprising a p73 OD.

6. The nanoparticle of claim 1, wherein the nanoparticle comprises:
a) a core comprising PEI and a polynucleotide encoding p53; and
b) a glucose/PEG conjugate surrounding the core of (a).

7. The nanoparticle of claim 1, wherein the nanoparticle comprises:
a) a core comprising PEI and a polynucleotide encoding a p53 chimera comprising a p73 OD; and
b) a glucose/PEG conjugate surrounding the core of (a).

8. A method of delivering a nanoparticle to a cell, comprising contacting the cell with the nanoparticle of claim 1 under conditions whereby the nanoparticle binds to a glucose transporter at the cell surface and is internalized by the cell.

9. The method of claim 8, wherein the cell is in vivo.

10. The method of claim 8, wherein the cell is in vitro.

11. A method of delivering an active agent to a tumor cell in a subject in need thereof, comprising delivering the nanoparticle of claim 1 to the subject, whereby the nanoparticle binds to a glucose transporter at the tumor cell surface and is internalized by the tumor cell, thereby delivering the active agent to the tumor cell.

12. A method of decreasing the size of a tumor in a subject in need thereof, comprising delivering an effective amount of the nanoparticle of claim 1 to the subject, whereby the nanoparticle binds to a glucose transporter on the surface of a tumor cell and is internalized, thereby decreasing the size of the tumor in the subject.

13. A method of treating cancer in a subject in need thereof, comprising delivering an effective amount of the nanoparticle of claim 5 to the subject, thereby treating cancer in the subject.

14. The method of claim 13, further comprising the step of administering to the subject a chemotherapeutic agent, an anti-angiogenic agent, a cytokine, a hormone, a radiation treatment, a surgical treatment or any combination thereof.

15. The method of claim 14, wherein the further step is carried out before, after and/or simultaneously with the delivery of the nanoparticles to the subject.

16. A composition comprising the nanoparticle of claim 1 and a pharmaceutically acceptable carrier.

17. A kit comprising the nanoparticle of claim 1.

18. An in vitro method of diagnosing cancer in a subject, comprising:
a) contacting the nanoparticle of claim 1 with cells from the subject, wherein the nanoparticle comprises a detectable marker;
b) measuring the rate and/or amount and/or specificity of internalization of the nanoparticles into the cells of the subject; and
c) comparing the rate and/or amount and/or specificity of internalization of the nanoparticles into the cells of the subject with the rate and/or amount and/or specificity of internalization of the nanoparticles into cells of a control subject and/or control cells from the subject being diagnosed, whereby an increase in the amount and/or rate and/or specificity of internalization of the nanoparticles into cells of the subject as compared with the cells of the control subject and/or the control cells is diagnostic of cancer in the subject.

19. An in vivo method of diagnosing cancer in a subject, comprising:
a) delivering a nanoparticle of claim 1 to the subject, wherein the nanoparticle comprises an imaging agent;
b) detecting a signal from the imaging agent in the subject; and
c) comparing the signal from the imaging agent in the subject with the signal from the same imaging agent in a control subject and/or in control tissue/cells of the subject being diagnosed, whereby an alteration in the signal from the subject as compared with the signal from the control subject and/or control tissue/cells is diagnostic of cancer in the subject.

20-30. (canceled)
